(19) United States
(12) Patent Application Publication
(10) Pub. No.: US 2020/0323950 A1
(43) Pub. Date: Oct. 15, 2020

(54) ANTIBACTERIAL PEPTIDES AND COMBINATIONS FOR CO-THERAPY

(71) Applicant: ARREVUS, INC., RALEIGH, NC (US)

(72) Inventors: Carl N. KRAUS, Raleigh, NC (US); Laszlo OTVOS, Audubon, PA (US)

(21) Appl. No.: 16/954,230

(22) PCT Filed: Dec. 17, 2018
(86) PCT No.: PCT/US2018/066002

§ 371 Date: Jun. 16, 2020

Related U.S. Application Data
(60) Provisional application No. 62/599,828, filed on Dec. 18, 2017.
(60) Provisional application No. 62/697,001, filed on Jul. 12, 2018.

(51) Int. Cl. A61K 38/16; A61P 31/04; A61K 38/12; A61K 45/06

(52) U.S. Cl. CPC A61K 38/16; A61K 45/06; A61K 38/12; A61P 31/04

(57) ABSTRACT

Described herein are combinations of antibacterial peptides with antibiotics. Also described herein are methods for using the combinations to increase the sensitivity of antibiotic resistant bacteria to an antibiotic, widen the therapeutic index of the antibacterial peptides, and treat bacterial infections in a subject, patient, or other host animal.

### CROSS-REFERENCE TO RELATED APPLICATIONS

This application claims priority under 35 U.S.C. § 119(e) to U.S. Provisional Application No. 62/599,828, filed on Dec. 18, 2017; and to U.S. Provisional Application No. 62/697,001, filed Jul. 12, 2018, the entire disclosure of each of which is incorporated herein by reference.

### TECHNICAL FIELD

The invention described herein relates to combinations of antibacterial peptides with antibiotics. The invention also relates to methods for using the combination to increase the sensitivity of antibiotic resistant bacteria to an antibiotic, widen the therapeutic index of the antibacterial peptides, and treat bacterial infections in a subject, patient, or other host animal.

### BACKGROUND AND SUMMARY OF THE INVENTION

Antibiotic resistance of microorganism is a growing problem worldwide. The improper use and overuse of currently available antibiotics has contributed to the problem. The development of new antibiotics and alternative methods of treating infections is needed. In addition, there is a need for compounds and combinations of compounds that can overcome, evade, or undo the desensitization achieved by resistant bacteria to conventional therapies, lower the resistance of antibiotic-resistant microorganisms, and treat antibiotic-resistant infections.

Proline-rich peptide dimers, such as A3-APO, have been reported to exhibit antibacterial properties. Otvos et al., J. Med. Chem. 48:5349 (2005).

It has been discovered that although such proline-rich peptides are effective by one or more specific modes of action, when used in combination with various other antibiotics, the activity of proline-rich peptides is more generalized. In other words, it has been surprisingly found that proline-rich peptides can be paired with a wide variety of other antibiotics that have unrelated modes of action, and the resulting combination exhibits unexpectedly high potency against bacteria, especially resistant bacteria. In certain instances, synergy is observed by administering a combination of two or more active pharmaceutical ingredients, only with certain combinations because the different modes of action of the two or more active pharmaceutical ingredients must cooperate in specific ways.

However, it has been unexpectedly observed that proline-rich peptide dimers, such as A3-APO, analogs and derivatives thereof, oligomers thereof, and/or salts of any of the foregoing exhibit synergy with a wider variety of companion antibiotics. Without being bound by theory, it is believed herein that proline-rich peptide dimers, such as A3-APO, analogs and derivatives thereof, oligomers thereof, and/or salts thereof exhibit synergy with a wider variety of companion antibiotics because the proline-rich peptide dimers are not restricted to specific combinations of modes of action. Instead, it is believed herein that they capitalize on the more generalized under-stress conditions that are created by administering the other antibiotic, where, for example, errors in protein folding cannot be overcome. That capitalization is very general and results in improved outcomes against a wide variety of diverse pathogens, even when the resistance mechanisms of those pathogens arise from unrelated defensive mechanisms. Thus, the co-administration of A3-APO, analogs and derivatives thereof, oligomers thereof, and/or salts thereof may more generally increase potency, widen the therapeutic index of the other antibiotic, and/or re-sensitize or increase the sensitivity of the pathogen to the other antibiotic.

In one illustrative embodiment of invention described herein, a composition comprising A3-APO or analogs, derivatives, or oligomers thereof, or a pharmaceutically acceptable salt thereof and an antibiotic is described.

In another embodiment, a unit dose comprising A3-APO or analogs, derivatives, or oligomers thereof, or a pharmaceutically acceptable salt thereof and an antibiotic is described.

In another embodiment, a kit comprising A3-APO or analogs, derivatives, or oligomers thereof, or a pharmaceutically acceptable salt thereof and an antibiotic, and instructions for administering the compounds is described.

In another embodiment, a method of treating a bacterial infection in a subject, patient, or other host animal in need thereof, comprising administering to the subject, patient, or other host animal a therapeutically effective dose of A3-APO or analogs, derivatives, or oligomers thereof, or a pharmaceutically acceptable salt thereof and an antibiotic is described.

In another embodiment, a method of increasing the sensitivity of antibiotic resistant bacteria to an antibiotic, comprising contacting the bacteria with A3-APO or analogs, derivatives, or oligomers thereof, or a pharmaceutically acceptable salt thereof and the antibiotic is described.

In another embodiment, a method of widening the therapeutic index of A3-APO or analogs, derivatives, or oligomers thereof, for treatment of a bacterial infection in a subject, patient, or other host animal, comprising administering to the subject, patient, or other host animal a therapeutically effective dose of A3-APO or analogs, derivatives, or oligomers thereof, or a pharmaceutically acceptable salt thereof and an antibiotic is described.

In another embodiment, a method of reducing the risk of recurrence of a microorganism, e.g., bacteria, infection in a subject, patient, or other host animal in need thereof, comprising administering to the subject, patient, or other host animal having a bacterial infection a therapeutically effective dose of A3-APO or analogs, derivatives, or oligomers thereof, or a pharmaceutically acceptable salt thereof and an antibiotic is described.

### BRIEF DESCRIPTION OF THE DRAWINGS

FIGS. 1A and 1B show synergy between peptide A3-APO (MIC 32-64 mg/L) and (A) imipenem (MIC>256 mg/L) and (B) colistin (MIC 64 mg/L) against the multidrug-resistant Klebsiella pneumoniae K97/09 strain in vitro. The plus signs indicate visible bacterial growth in the wells, the shaded boxes indicate absence of visible bacterial growth at doses substantially lower than the MIC values for the compounds alone. The antibiotics and the peptide were applied to bacteria in mid-log growing phase concomitantly.

FIGS. 2A-2B shows efficacy of synergistic combinations of peptide A3-APO and human equivalent doses of colistin or imipenem in a mouse model of systemic Klebsiella pneumoniae infection as read by survival (A) and blood bacterial counts (B) data. Treatment was administered at 1, 12 and 24 h after infection. Imipenem (30 mg/kg) and colistin (10 mg/kg) were administered subcutaneously (sc). Peptide A3-APO was added intramuscularly (im) at doses 1 or 0.5 mg/kg. Survival was monitored continuously after infection. The open symbols in B represent cfu/mL counts from the blood of individual mice, the filled magenta circle is a mean of the individual mouse data. The detection limit of this assay is 1000 cfu/mL, all results under this value are displayed as 100 for simplicity.

FIGS. 3A-3B shows efficacy of synergistic combinations of peptide A3-APO and a significantly reduced dose of colistin in a mouse model of systemic Klebsiella pneumoniae infection as read by survival (A) and blood bacterial counts (B) data. Treatment was administered at 1 and 12 h after infection. Colistin at a 1 mg/kg dose was administered subcutaneously (sc). Peptide A3-APO was added intramuscularly (im) at doses 1 or 0.5 mg/kg. Survival was monitored after 12, 24 and 36 h of infection. The open symbols in B represent cfu/mL counts from the blood of individual mice, the filled magenta circles (4 h) or filled purple triangles (11 h) are means of the individual mouse data. The detection limit of this assay is 1000 cfu/mL, all results under this value are displayed as 100 for simplicity. The broken purple lines in the colistin alone and colistin—low A3-APO columns at the 11 h sampling is a proxy of ensuing survival. Almost all mice (with the exception of a single mouse treated with 1 mg/kg A3-APO alone) of this assay when the 11 h bacterial load was above 4000 cfu/mL succumbed to K. pneumoniae infection irrespective of the second antibacterial dosing.

FIG. 4 shows the excitation and emission spectra of DTM-linked tetrameric PrAMP in MilliQ water.

FIG. 5 shows the translational diffusion coefficients of PrAMPs and dioxane, as internal reference for hydrodynamic radius, measured in ²H₂O at 298 K. The translational diffusion-induced signal attenuation in the presence of pulsed-field gradients for PrAMPs (▴ dimer, ▾ tetramer-butene-NHNH₂, ▪ tetramer-bismal-NHNH₂, ♦ tetramer-xylene-NHNH₂, [symbol] tetramer-DTM-NHNH₂) and dioxane (open symbols) are shown as relative intensities versus the strength of the diffusion encoding, γ²s²g²δ²(Δ-δ/3-τ/2). Lines represent the results of non-linear regression to Eq. 1. Resultant diffusion coefficients of the non-linear regression are listed in Table 1.

FIG. 6A shows synergy between peptide A3-APO (MIC 32 mg/L) and imipenem (MIC 64 mg/L) against multidrug-resistant Acinetobacter baumannii BAA-1605 (ABC) in vitro. The plus signs indicate visible bacterial growth in the wells, the shaded boxes indicate absence of visible bacterial growth at doses substantially lower than the MIC values for the compounds alone. The antibiotics and the peptides were applied to bacteria in mid-log growing phase concomitantly.

FIGS. 6B and 6C show synergy between peptide A3-APO (MIC 32 mg/L) and (B) imipenem (MIC>256 mg/L) and (C) colistin (MIC 64 mg/L) against multidrug-resistant Klebsiella pneumoniae K97/09 (KPC) in vitro. The plus signs indicate visible bacterial growth in the wells, the shaded boxes indicate absence of visible bacterial growth at doses substantially lower than the MIC values for the compounds alone. The antibiotics and the peptides were applied to bacteria in mid-log growing phase concomitantly.

FIG. 7 shows efficacy of peptide A3-APO as a monotherapy and synergistic combinations of A3-APO results in a reduced dose of colistin and a survival advantage in a bacteremia mouse model of Klebsiella pneumoniae (survival, panel A and blood CFU, panel B). Treatment was administered at 1 and 12 h after infection as described in Materials and Methods. Colistin at a 1 mg/kg dose was administered subcutaneously (sc). Peptide A3-APO was added intramuscularly (im) at doses of 1 or 0.5 mg/kg. Survival was monitored after 12, 24 and 36 h of infection. The numbers in parenthesis in panel A indicate the number of surviving animals at 36 h (out of 5). The open symbols in panel B represent CFU/mL counts from the blood of individual mice collected 6 h after infection, the filled magenta circle is a mean of the individual mouse data. The detection limit of our assay is 103 CFU/mL, all results under this value are displayed as 100.

FIG. 8 shows combinations of peptide A3-APO and colistin or imipenem in a bacteremia mouse model with Klebsiella pneumoniae infection with survival (panel A) and blood bacterial counts (panel B) outcomes. Treatment was administered at 1, 12 and 24 h after infection as described in Materials and Methods. Imipenem (30 mg/kg) and colistin (10 mg/kg) were administered subcutaneously. Peptide A3-APO was added intramuscularly at doses 1 or 0.5 mg/kg. Survival was monitored continuously after infection. The Panel A figures in parenthesis represent the number of surviving animals after 36 h (out of 5). The open symbols in panel B represent CFU/mL counts from the blood of individual mice, and the filled magenta circle is a mean of the individual mouse data. The assay detection limit is 103 CFU/mL; all results under this value are displayed as 100.

### DETAILED DESCRIPTION

Several illustrative embodiments of the invention are described by the following clauses:

A composition comprising A3-APO or an analog, derivative, or oligomer thereof, or a pharmaceutically acceptable salt of the foregoing; and an antibiotic.

A unit dose comprising A3-APO or an analog, derivative, or oligomer thereof, or a pharmaceutically acceptable salt thereof; and an antibiotic.

A kit comprising A3-APO or an analog, derivative, or oligomer thereof, or a pharmaceutically acceptable salt thereof; an antibiotic; and instructions for co-administering the A3-APO or an analog, derivative, or oligomer thereof, or a pharmaceutically acceptable salt thereof, and the antibiotic as part of a method for treating an infection.

The composition, unit dose, or kit of any one of the preceding clauses wherein the A3-APO or analog, derivative, or oligomer thereof, or a pharmaceutically acceptable salt thereof, and the antibiotic are each present in an amount individually subtherapeutic.

The composition, unit dose, or kit of any one of the preceding clauses wherein the dose of the A3-APO or an analog, derivative, or oligomer thereof, or a pharmaceutically acceptable salt thereof is within the hermetic zone or range.

The composition, unit dose, or kit of any one of the preceding clauses wherein the A3-APO or an analog, derivative, or oligomer thereof, or a pharmaceutically acceptable salt thereof, and the antibiotic are present in an amount in combination adapted for inhibiting toxin production by bacteria.

A method for treating a bacterial infection in a host animal, the method comprising administering to the host animal a therapeutically effective dose of a combination of A3-APO or analog, derivative, or oligomer thereof, or a pharmaceutically acceptable salt thereof; and an antibiotic.

A method for increasing the sensitivity of antibiotic resistant bacteria to an antibiotic, the method comprising contacting the bacteria with a therapeutically effective amount of A3-APO or analog, derivative, or oligomer thereof, or a pharmaceutically acceptable salt thereof.

A method for widening the therapeutic index of an antibiotic for treating a bacterial infection in a host animal, the method comprising administering to the host animal a therapeutically effective amount of A3-APO or analog, derivative, or oligomer thereof, or a salt thereof.

A method for reducing the risk of recurrence of a bacterial infection in a host animal, the method comprising administering to the host animal a therapeutically effective amount of A3-APO or analog, derivative, or oligomer thereof, or a pharmaceutically acceptable salt thereof.

The composition, unit dose, kit, or method of any one of the preceding clauses wherein the A3-APO or analog, derivative, or oligomer thereof, or a pharmaceutically acceptable salt thereof, and the antibiotic are each present in an amount individually subtherapeutic.

The composition, unit dose, kit, or method of any one of the preceding clauses wherein the dose of the A3-APO or an analog, derivative, or oligomer thereof, or a pharmaceutically acceptable salt thereof is within the hermetic zone or range.

The composition, unit dose, kit, or method of any one of the preceding clauses wherein the A3-APO or an analog, derivative, or oligomer thereof, or a pharmaceutically acceptable salt thereof, and the antibiotic are present in an amount in combination adapted for inhibiting toxin production by bacteria.

The composition, unit dose, kit, or method of any one of the preceding clauses wherein the A3-APO or analog, derivative, or oligomer thereof, or a pharmaceutically acceptable salt thereof and the antibiotic are administered simultaneously.

The composition, unit dose, kit, or method of any one of the preceding clauses wherein the A3-APO or analog, derivative, or oligomer thereof, or a pharmaceutically acceptable salt thereof and the antibiotic are administered sequentially.

The composition, unit dose, kit, or method of any one of the preceding clauses wherein the host animal has or is at risk of having resistant bacteria.

The composition, unit dose, kit, or method of any one of the preceding clauses wherein the A3-APO or analog, derivative, or oligomer thereof, or a pharmaceutically acceptable salt thereof is administered to the host animal prior to the antibiotic to prime treatment with the antibiotic.

The composition, unit dose, kit, or method of any one of the preceding clauses wherein the infection is or is at risk of being refractory to treatment of the bacterial infection with the antibiotic alone.

The composition, unit dose, kit, or method of any one of the preceding clauses wherein the infection is caused at least in part by Gram-negative bacteria.

The composition, unit dose, kit, or method of any one of the preceding clauses wherein the infection is caused at least in part by E. coli, Klebsiella, Acinetobacter, Pseudomonas, Burkholderia, or a combination thereof.

The composition, unit dose, kit, or method of any one of the preceding clauses wherein the infection is caused at least in part by Pseudomonas, including Pseudomonas aeruginosa, Staphylococcus, including Staphylococcus aureus, Enterococcus, including Enterococcus faecium, Clostridium, including Clostridium difficile, or a combination thereof.

The composition, unit dose, kit, or method of any one of the preceding clauses wherein the infection is caused at least in part by Neisseria gonorrhoeae.

The composition, unit dose, kit, or method of any one of the preceding clauses wherein the infection is caused at least in part by Mycobacterium tuberculosis.

The composition, unit dose, kit, or method of any one of the preceding clauses wherein the infection is caused at least in part by Coxiella.

The composition, unit dose, kit, or method of any one of the preceding clauses wherein infection is caused at least in part by bacteria resistant to one or more beta-lactam antibiotics, quinolones or fluoroquinolones, gentamicin or analog or derivative thereof, polymyxin, or a combination thereof.

The composition, unit dose, kit, or method of any one of the preceding clauses wherein the infection is caused at least in part by Pseudomonas resistant to one or more polymyxins.

The composition, unit dose, kit, or method of any one of the preceding clauses wherein the infection is caused at least in part by Pseudomonas resistant to colistin.

The composition, unit dose, kit, or method of any one of the preceding clauses wherein the infection is caused at least in part by one or more multi-drug resistant bacteria.

The composition, unit dose, kit, or method of any one of the preceding clauses wherein the infection is caused at least in part by carbapenem-resistant Enterobacteriaceae (CRE).

The composition, unit dose, kit, or method of any one of the preceding clauses wherein the infection is caused at least in part by extended spectrum beta-lactamase (ESBL) producing bacteria.

The composition, unit dose, kit, or method of any one of the preceding clauses wherein the infection is caused at least in part by carbapenem-resistant E. coli, carbapenem-resistant Klebsiella, or a combination thereof. CRE, and more specifically carbapenem-resistant E. coli and carbapenem-resistant Klebsiella are reportedly the clinically challenging infections to treat.

The composition, unit dose, kit, or method of any one of the preceding clauses wherein the infection is anthrax.

The composition, unit dose, kit, or method of any one of the preceding clauses wherein the infection is tularemia.

The composition, unit dose, kit, or method of any one of the preceding clauses wherein the infection is a central nervous system (CNS) infection.

The composition, unit dose, kit, or method of any one of the preceding clauses wherein the infection is a nosocomial infection.

The composition, unit dose, kit, or method of any one of the preceding clauses wherein the infection is a community infection.

The composition, unit dose, kit, or method of any one of the preceding clauses wherein the infection is a urinary tract infection.

The composition, unit dose, kit, or method of any one of the preceding clauses wherein the infection is a complicated urinary tract infection.

The composition, unit dose, kit, or method of any one of the preceding clauses wherein the infection is melioidosis.

The composition, unit dose, kit, or method of any one of the preceding clauses wherein the infection is bacteremia.

The composition, unit dose, kit, or method of any one of the preceding clauses wherein the infection is a wound infection.

The composition, unit dose, kit, or method of any one of the preceding clauses wherein the infection is an infection associated with a prosthetic.

The composition, unit dose, kit, or method of any one of the preceding clauses wherein the infection is associated with a device, such as a ventilator, urinary catheter, or intravenous catheter.

The composition, unit dose, kit, or method of any one of the preceding clauses wherein the infection is HVAP pneumonia.

The composition, unit dose, kit, or method of any one of the preceding clauses wherein the antibiotic is a polymyxin.

The composition, unit dose, kit, or method of any one of the preceding clauses wherein the antibiotic is colistin.

The composition, unit dose, kit, or method of any one of the preceding clauses wherein the antibiotic is a beta-lactam antibiotic.

The composition, unit dose, kit, or method of any one of the preceding clauses wherein the antibiotic is a carbapenem.

The composition, unit dose, kit, or method of any one of the preceding clauses wherein the antibiotic is imipenem.

Unless otherwise defined, all technical and scientific terms used herein have the same meaning as commonly understood by one of skill in the art to which this invention belongs. The terminology used in the description of the invention herein is for the purpose of describing certain embodiments herein, and is not intended to be limiting of the invention. All publications, patent applications, patents, patent publications and other references cited herein are incorporated by reference in their entireties for the teachings relevant to the sentence and/or paragraph in which the reference is presented.

Naturally occurring amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

As used in the description of the invention and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

The transitional phrase “consisting essentially of” means that the scope of a claim is to be interpreted to encompass the specified materials or steps recited in the claim, and those that do not materially affect the basic and novel characteristics of the claimed invention.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In another embodiment, the inhibition or reduction results in little or essentially no detectible activity (at most, an insignificant amount, e.g., less than about 10% or even 5%).

A “therapeutically effective” amount as used herein is an amount that provides some improvement or benefit to the subject, patient, or other host animal. Alternatively stated, a “therapeutically effective” amount is an amount that will provide some alleviation, mitigation, or decrease in at least one clinical symptom in the subject, patient, or other host animal. Those skilled in the art will appreciate that the therapeutic effects need not be complete or curative, as long as some benefit is provided to the subject, patient, or other host animal.

By the terms “treat,” “treating,” or “treatment of” it is intended that the severity of the subject's condition is reduced or at least partially improved or modified and that some alleviation, mitigation or decrease in at least one clinical symptom is achieved.

A “synergistic” effect, as used herein, is an effect that is greater than additive when two molecules are administered to a subject, patient, or other host animal simultaneously or sequentially. It is to be understood that sequential administration may be in any order. It is also to be understood that sequential administration may include variations where a time delay between the co-administration steps is included within the co-administration dosing window.

The term “therapeutic index,” as used herein, refers to the ratio of the dose of drug that causes adverse effects at an incidence/severity not compatible with the targeted indication (e.g., toxic dose in 50% of subjects, TD50) to the dose that leads to the desired pharmacological effect (e.g., efficacious dose in 50% of subjects, ED50). A widening of the therapeutic index refers to an increase in the difference between the toxic and therapeutic dose.

The term “antibiotic-resistant,” as used herein, refers to the ability of a microorganism to resist the toxic effects of an antibiotic. Resistance typically occurs when the microorganism produces one or proteins or other components that can disable an antibiotic or prevents transport of the antibiotic into the cell. As used herein, the term also includes microorganisms that undergo reversal of tolerance. An antibiotic-resistant microorganism is one in which the minimum inhibitory concentration (MIC) is increase by at least 10% relative to the average MIC of the non-resistant strain.

The term “minimum inhibitory concentration (MIC)” refers to the lowest concentration of a compound or molecule that prevents visible growth of a bacterium.

The term “sequentially” refers to the administration of two or more agents one after the other and close enough in time that each of the agents exerts a biological activity on the other agent, e.g., the two or more agents have an effect in combination.

The bacterial target of A3-APO is the C-terminal D-E helix of the 70 kDa bacterial heat shock protein DnaK (Otvos et al., J. Med. Chem. 48:5349 (2005); Kragol et al., Biochemistry 40:3016 (2001); Bikker et al., Chem. Biol. Drug Des. 68:148 (2006)). Because DnaK is present in all bacteria, A3-APO is effective against a wide variety of bacteria and may be effective in combination with all classes of antibiotics.

Illustrative antibacterial peptides in A3-APO, which is a peptide dimer having the following structure:

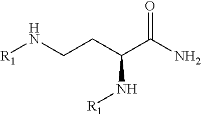

wherein R₁ is Chex-RPDKPRPYLPRPRPPRPVR, and Chex is 1-amino-cyclohexyl carboxylic acid.

Additional illustrative antibacterial peptides include analogs, derivatives, and oligomers of A3-APO.

| | | Yield |
|---|---|---|
| Antibacterial Peptide | Structure* | (overall) |
| A3APO¹⁴ | 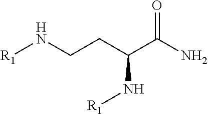 | 30% |
| dimer-NHNH₂ | 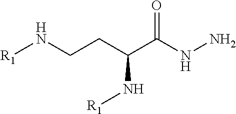 | 25% |
| tetramer-DTM-ol | 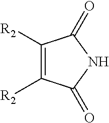 | 12.3% |
| tetramer-DTM-NH₂ | 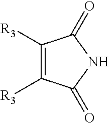 | 15.9% |
| tetramer-DTM-NHNH₂ | 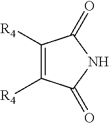 | 16.6% |
| tetramer-butene-NHNH₂ | 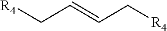 | 16.1% |
| tetramer-bismal-NHNH₂ | 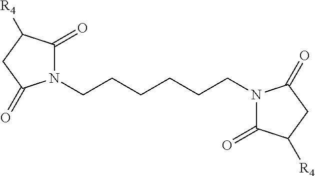 | 13.3% |
| tetramer-xylene-NHNH₂ | 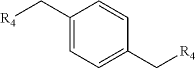 | 15.0% |

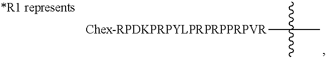

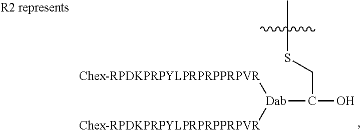

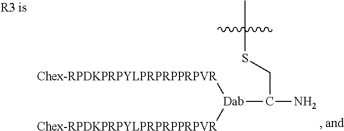

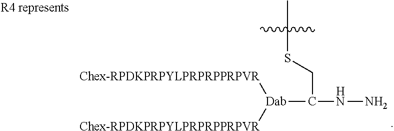

The peptide chains of A3-APO contain acidic or basic groups (such as amine or carboxyl groups) and therefore it is to be understood that such groups can be optionally in the free base form or a salt form. All references to A3-APO, unless indicated otherwise, also refer to various salt forms of the molecule, such as pharmaceutically acceptable salts.

The term “salts” includes addition salts of free acids or free bases. The term “pharmaceutically acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, .beta.-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from A3-APO by reacting, for example, the appropriate acid or base with A3-APO.

One aspect of the invention relates to a composition comprising A3-APO or analogs, derivatives, or oligomers thereof, or a pharmaceutically acceptable salt thereof and an antibiotic. The composition may comprise more than one antibiotic, e.g., 1, 2, 3, 4, or 5 or more antibiotics.

The composition may be a dosage form, e.g., a unit dosage form. In another embodiment, the A3-APO or analogs, derivatives, or oligomers thereof, or a pharmaceutically acceptable salt thereof and antibiotic are both present in therapeutically effective amounts. In another embodiment, the A3-APO or analogs, derivatives, or oligomers thereof, and antibiotic are both present in synergistic amounts, e.g., amounts that, when administered to a subject, patient, or other host animal, will produce a synergistic effect.

In another embodiment, the A3-APO or analogs, derivatives, or oligomers thereof, or a pharmaceutically acceptable salt thereof and/or the antibiotic is present in an amount that, by itself, is not therapeutic but produces a therapeutic effect in combination. In another embodiment, the A3-APO or analogs, derivatives, or oligomers thereof, or a pharmaceutically acceptable salt thereof and/or the antibiotic is present in an amount that, by itself, is not therapeutic but renders a third antibiotic therapeutically effective.

In another embodiment, the relative amount of the A3-APO or analogs, derivatives, or oligomers thereof, or a pharmaceutically acceptable salt thereof is minimized. It has been unexpectedly observed that excessively high doses of the A3-APO or analogs, derivatives, or oligomers thereof, or a pharmaceutically acceptable salt thereof either do not provide a dose-response related improvement in outcome over the antibiotic alone and lower doses of the A3-APO or analogs, derivatives, or oligomers thereof, or a pharmaceutically acceptable salt thereof. In other cases, excessively high doses of the A3-APO or analogs, derivatives, or oligomers thereof, or a pharmaceutically acceptable salt thereof provide an inverted dose-response related therapeutic outcome, or biphasic dose response, leading to hormesis. Accordingly, in another embodiment, the dose of the A3-APO or analogs, derivatives, or oligomers thereof, or a pharmaceutically acceptable salt thereof is below a pre-determined threshold, and/or within the hermetic zone or range, as described herein.

In another embodiment, a co-therapy is described where the inclusion of A3-APO or analogs, derivatives, or oligomers thereof, or a pharmaceutically acceptable salt thereof provides an expanded therapeutic window for the antibiotic. For example, at certain doses, colistin is reportedly nephrotoxic. The co-therapy methods described herein may increase the therapeutic window for colistin and other antibiotics providing for lower dosing protocols of colistin, and other antibiotics while maintaining therapeutic efficacy.

Another aspect of the invention relates to a method of increasing the sensitivity of antibiotic resistant microorganisms, e.g., bacteria, to an antibiotic, comprising contacting the bacteria with A3-APO or analogs, derivatives, or oligomers thereof, or a pharmaceutically acceptable salt thereof. The method may further comprise contacting the bacteria with the antibiotic.

A further aspect of the invention relates to a method of widening the therapeutic index of A3-APO or analogs, derivatives, or oligomers thereof, for treatment of a microorganism, e.g., bacteria, infection in a subject, patient, or other host animal, comprising administering to the subject, patient, or other host animal a therapeutically effective dose of A3-APO or analogs, derivatives, or oligomers thereof, or a pharmaceutically acceptable salt thereof and an antibiotic. In another embodiment, the therapeutic index is widened by at least about 5%, e.g., at least about 10%, 20%, 50%, 100%, 200%, 500%, or more.

An additional aspect of the invention relates to a method of treating a microorganism, e.g., bacteria, infection in a subject, patient, or other host animal in need thereof, comprising administering to the subject, patient, or other host animal a therapeutically effective dose of A3-APO or analogs, derivatives, or oligomers thereof, or a pharmaceutically acceptable salt thereof and an antibiotic.

Another aspect of the invention relates to a method of reducing the risk of recurrence of a microorganism, e.g., bacteria, infection in a subject, patient, or other host animal in need thereof, comprising administering to the subject, patient, or other host animal having a bacterial infection a therapeutically effective dose of A3-APO or analogs, derivatives, or oligomers thereof, or a pharmaceutically acceptable salt thereof and an antibiotic.

In each of the methods of the invention, the A3-APO or analogs, derivatives, or oligomers thereof, or a pharmaceutically acceptable salt thereof and the antibiotic may be administered simultaneously, e.g., in the same dosage form or in separate dosage forms. It has been unexpectedly discovered that pre-administration of the A3-APO or analogs, derivatives, or oligomers thereof, or a pharmaceutically acceptable salt thereof provides a therapeutic improvement in outcome, where the antibiotic is administered second, and optionally after a short time delay. In each of the methods of the invention, the A3-APO or analogs, derivatives, or oligomers thereof, or a pharmaceutically acceptable salt thereof and the antibiotic may be administered sequentially, but close enough together in time to exert a biological effect on each other. In another embodiment, the A3-APO or analogs, derivatives, or oligomers thereof, or a pharmaceutically acceptable salt thereof is administered before the antibiotic. In other embodiments, the antibiotic is administered before the A3-APO or analogs, derivatives, or oligomers thereof, or a pharmaceutically acceptable salt thereof. The second molecule may be administered at any effective time point after the first molecule is administered, e.g., about 1, 2, 3, 4, 5, 6, 7, 8, 9, 10, 15, 20, 25, 30, 35, 40, 45, 50, 55, or 60 minutes or about 1.5, 2, 3, 4, 5, 6, 7, 8, 9, 10, 11, or 12 hours after the first molecule is administered.

In another embodiment of the methods of the invention, the subject, patient, or other host animal is one that has been diagnosed as being infected with antibiotic-resistant bacteria or is suspected of being infected with antibiotic-resistant bacteria. The subject, patient, or other host animal may not have been previously treated for the infection. The subject, patient, or other host animal may be administered A3-APO or analogs, derivatives, or oligomers thereof, or a pharmaceutically acceptable salt thereof first to prime the subject, patient, or other host animal for antibiotic treatment.

In another embodiment, the subject, patient, or other host animal has been treated with an antibiotic and the treatment has been ineffective (e.g., the infection has not been reduced, has been reduced but not eradicated, or was thought to have been eradicated but has returned). A3-APO or analogs, derivatives, or oligomers thereof, or a pharmaceutically acceptable salt thereof may be added to the treatment with the same antibiotic or with a different antibiotic.

In another embodiment, the subject, patient, or other host animal may be immunocompromised or otherwise have diminished ability to fight the infection.

The methods of the present invention may permit the A3-APO or analogs, derivatives, or oligomers thereof, or a pharmaceutically acceptable salt thereof and/or the antibiotic to be administered at a dose that that would not be therapeutically effective if administered alone. The MIC of the A3-APO and analogs, derivatives, and oligomers thereof, and/or antibiotic when provided together may be decreased by at least about 5%, e.g., at least about 10%, 15%, 20%, 25%, 30%, 35%, 40%, 45%, 50%, or more.

The microorganism, e.g., bacteria, may be any microorganism known in the art. In another embodiment, the microorganism is an antibiotic-resistant strain. Pathogenic bacteria and other microorganisms include, but are not limited to, Rickettsia, Chlamydia, Mycobacteria, Clostridia, Corynebacteria, Mycoplasma, Ureaplasma, Legionella, Shigella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., Helicobacter spp., and any other pathogenic microorganism now known or later identified (see, e.g., Microbiology, Davis et al, Eds., 4^{th} ed., Lippincott, New York, 1990, the entire contents of which are incorporated herein by reference for the teachings of pathogenic microorganisms). Specific examples of microorganisms include, but are not limited to, Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum, Bacillus anthracis, Salmonella typhi, Vibrio cholera, Pasteurella pestis (Yersinia pestis), Pseudomonas aeruginosa, Campylobacter jejuni, Clostridium difficile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, and enterotoxic Escherichia coli.

Classes of antibiotics suitable for use in the present invention include, but are not limited to, aminoglycosides, antimicrobial peptides, carbapenems, cephalosporins, cephems, glycoproteins, fluoroquinolones/quinolones, oxazolidinones, penicillins, streptogramins, sulfonamides, and tetracyclines.

Aminoglycosides include, but are not limited to, amikacin, gentamycin, tobramycin, netromycin, streptomycin, kanamycin, paromomycin, and neomycin.

Antimicrobial peptides include, but are not limited to, colistin, topical polymyxin B, daptomycin, gramicidin, pexiganan (magainin), omiganan, iseganan, mefloquine, venturicidin A, antimycin, myxothiazol, stigmatellin, diuron, iodoacetamide, potassium tellurite hydrate, aDL-vinylglycine, N-ethylmaleimide, L-allyglycine, diaryquinoline, betaine aldehyde chloride, acivcin, psicofuraine, buthionine sulfoximine, diaminopemelic acid, 4-phospho-D-erythronhydroxamic acid, motexafin gadolinium and xycitrin.

Carbapenems include, but are not limited to, imipenem/cilastatin sodium, meropenem, ertapenem, and panipenem/betamipron.

Cephalosporins include, but are not limited to, cefixime, cefpodoxime, ceftibuten, cefdinir, cefaclor, cefprozil, loracarbef, cefadroxil, cephalexin, and cephradineze.

Cephems include, but are not limited to, cefepime, cefpirome, cefataxidime pentahydrate, ceftazidime, ceftriaxone, ceftazidime, cefotaxime, cefteram, cefotiam, cefuroxime, cefamandole, cefuroxime axetil, cefotetan, cefazolin sodium, cefazolin, and cefalexin.

Fluoroquinolones/quinolones include, but are not limited to, ciproflaxacin, levofloxacin, and ofloxacin, gatifloxacin, norfloxacin, lomefloxacin, trovafloxacin, moxifloxacin, sparfloxacin, gemifloxacin, and pazufloxacin.

Glycopeptides include, but are not limited to, include vancomycin, teicoplanin, and daptomycin.

Oxazolidinones include, but are not limited to, linezolid.

Penicillins include, but are not limited to, penicillin, amoxicillin, amoxicillin-clavulanate, ampicillin, ticarcillin, piperacillin-tazobactam, carbenicillin, piperacillin, mezocillin, benzathin penicillin G penicillin V potassium, methicillin, nafcillin, oxacillin, cloxacillin, and dicloxacillin.

Streptogramins include, but are not limited to, quinupristin/dafopristin and pristinamycin.

Sulphonamides include, but are not limited to, co-trimoxazole, sulfamethoxazole trimethoprim, sulfadiazine, sulfadoxine, and trimethoprim.

Tetracyclines include, but are not limited to, tetracycline, demeclocycline, minocycline, and doxycycline.

Other antimicrobial agents and antibiotics contemplated herein (some of which are listed above) include, but are not limited to; abrifam; acrofloxacin; aptecin, amoxicillin plus clavulonic acid; apalcillin; apramycin; astromicin; arbekacin; aspoxicillin; azidozillin; azlocillin; aztreonam; bacitracin; benzathine penicillin; benzylpenicillin; clarithromycin, carbencillin; cefaclor; cefadroxil; cefalexin; cefamandole; cefaparin; cefatrizine; cefazolin; cefbuperazone; cefcapene; cefdinir; cefditoren; cefepime; cefetamet; cefixime; cefinetazole; cefminox; cefoperazone; ceforanide; cefotaxime; cefotetan; cefotiam; cefoxitin; cefpimizole; cefpiramide; cefpodoxime; cefprozil; cefradine; cefroxadine; cefsulodin; ceftazidime; ceftriaxone; cefuroxime; cephalexin; chloramphenicol; chlortetracycline; ciclacillin; cinoxacin; clemizole penicillin; cleocin, cleocin-T, cloxacillin; corifam; daptomycin; daptomycin; demeclocycline; desquinolone; dibekacin; dicloxacillin; dirithromycin; doxycycline; enoxacin; epicillin; ethambutol; gemifloxacin; fenampicin; finamicina; fleroxacin; flomoxef; flucloxacillin; flumequine; flurithromycin; fosfomycin; fosmidomycin; fusidic acid; gatifloxacin; gemifloxaxin; isepamicin; isoniazid; josamycin; kanamycin; kasugamycin; kitasamycin; kalrifam, latamoxef; levofloxacin, levofloxacin; lincomycin; linezolid; lomefloxacin; loracarbaf; lymecycline; mecillinam; methacycline; methicillin; metronidazole; mezlocillin; midecamycin; minocycline; miokamycin; moxifloxacin; nafcillin; nafcillin; nalidixic acid; neomycin; netilmicin; norfloxacin; novobiocin; oflaxacin; oleandomycin; oxacillin; oxolinic acid; oxytetracycline; paromycin; pazufloxacin; pefloxacin; penicillin g; penicillin v; phenethicillin; phenoxymethyl penicillin; pipemidic acid; piperacillin and tazobactam combination; piromidic acid; procaine penicillin; propicillin; pyrimethamine; rifadin; rifabutin; rifamide; rifampin; rifapentene; rifomycin; rimactane, rofact; rokitamycin; rolitetracycline; roxithromycin; rufloxacin; sitafloxacin; sparfloxacin; spectinomycin; spiramycin; sulfadiazine; sulfadoxine; sulfamethoxazole; sisomicin; streptomycin; sulfamethoxazole; sulfisoxazole; quinupristan-dalfopristan; teicoplanin; temocillin; gatifloxacin; tetracycline; tetroxoprim; telithromycin; thiamphenicol; ticarcillin; tigecycline; tobramycin; tosufloxacin; trimethoprim; trimetrexate; trovafloxacin; vancomycin; verdamicin; azithromycin; and linezolid.

In another embodiment, the antibiotic is a polymyxin, such as colistin. In another embodiment, the antibiotic is a carbapenem, such as imipenem.

A3-APO and analogs, derivatives, and oligomers thereof, may be prepared by conventional and routine processes. For example, the peptide portion may be chemically synthesized using solid phase synthesis methods. In one method, the synthesis and analysis of peptides are described in Otvos et al., J. Med. Chem. 48:5349 (2005) and Cudic et al., Peptides 23:271 (2002).

The peptide may be made on a standard automated synthesizer. The peptide is detached from the resin and may be purified by reverse phase high pressure liquid chromatography. Matrix-assisted laser desorption/ionization (MALDI)-MS may be used to verify the accuracy of the sequences and their purity.

In one embodiment, the A3-APO or analogs, derivatives, or oligomers thereof, and antibiotic of the invention are administered directly to a subject, patient, or other host animal. In another embodiment, the compounds will be suspended in a pharmaceutically-acceptable carrier (e.g., physiological saline) and administered orally or by intravenous infusion, or administered subcutaneously, intramuscularly, intrathecally, intraperitoneally, intrarectally, intravaginally, intranasally, intragastrically, intratracheally, or intrapulmonarily. In another embodiment, the intratracheal or intrapulmonary delivery can be accomplished using a standard nebulizer, jet nebulizer, wire mesh nebulizer, dry powder inhaler, or metered dose inhaler. They can be delivered directly to the site of the disease or disorder, such as lungs, kidney, or intestines. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of antibiotics available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-; 20-, 50-, 100-, 150-, or more fold). Encapsulation of the A3-APO and analogs, derivatives, and oligomers thereof, and antibiotic in a suitable delivery vehicle (e.g., polymeric microparticles or implantable devices) may increase the efficiency of delivery, such as for oral delivery.

Another aspect of the invention relates to a kit comprising A3-APO or analogs, derivatives, or oligomers thereof, and an antibiotic and useful for carrying out the methods of the invention. The A3-APO or analogs, derivatives, or oligomers thereof, and antibiotic may be in the same container or in separate containers. The kit may further comprise additional reagents for carrying out the methods (e.g., buffers, containers, additional therapeutic agents) as well as instructions.

As a further aspect, the invention provides pharmaceutical formulations and methods of administering the same to achieve any of the therapeutic effects (e.g., treatment of infection) discussed above. The pharmaceutical formulation may comprise any of the reagents discussed above in a pharmaceutically acceptable carrier.

The term “pharmaceutically acceptable” generally refers to a material that is not biologically or otherwise undesirable, i.e., the material can be administered to a subject, patient, or other host animal without causing any undesirable biological effects such as toxicity.

The formulations of the invention can optionally comprise medicinal agents, pharmaceutical agents, carriers, adjuvants, dispersing agents, diluents, and the like.

The compounds of the invention can be formulated for administration in a pharmaceutical carrier in accordance with known techniques. See, e.g., Remington, The Science and Practice of Pharmacy (21^{st} Ed. 2006). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

A further aspect of the invention is a method of treating subjects in vivo, comprising administering to a subject, patient, or other host animal a pharmaceutical composition comprising A3-APO or analogs, derivatives, or oligomers thereof, and an antibiotic in a pharmaceutically acceptable carrier, wherein the pharmaceutical composition is administered in a therapeutically effective amount. Administration of the compounds of the present invention to a human subject or an animal in need thereof can be by any means known in the art for administering compounds.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into the brain for delivery to the central nervous system, into the pancreas, or into a tumor or the tissue surrounding a tumor). The most suitable route in any given case will depend on the nature and severity of the condition being treated and on the nature of the selected compound which is being used.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject, patient, or other host animal. The unit dosage form typically comprises from about 1 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compounds. Suitable formulations comprise citrate or bis/tris buffer (pH 6) or ethanol/water and contain from 0.1 to 0.2M of the compound.

The compound can alternatively be formulated for nasal administration or otherwise administered to the lungs of a subject, patient, or other host animal by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. For example, useful emulsifying agents include phosphatidyl cholines and lecithin.

In another embodiment, the compound is administered to the subject, patient, or other host animal in a therapeutically effective amount, as that term is defined above. Dosages of pharmaceutically active compounds can be determined by methods known in the art, see, e.g., Remington's Pharmaceutical Sciences (Maack Publishing Co., Easton, Pa.). The therapeutically effective dosage of any specific compound will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. For example, dosages are about 1 μmol/kg to 50 μmol/kg, and more specifically to about 22 μmol/kg and to 33 μmol/kg of the compound for intravenous or oral administration, respectively.

In another embodiment, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults.

The invention is described further in the following examples that are intended as illustrative only and should not be construed as limiting the invention.

### Examples

MATERIALS. Unless otherwise indicated, reagents and other materials are used as obtained from commercial suppliers. The colistin sulfate preparation (15,000 IU/mg) is obtained from Sigma-Aldrich Kft (Budapest, Hungary) and imipenem from MSD Budapest, Hungary Merck (tienamycin-formamidinemonohydrate sodium cilistatin marketed as Tienam).

BACTERIAL STRAINS. The K. pneumoniae strain originated from a human wound infection at Miskolc Healthcare Center/Semmelweis University Hospital and is designated as K97/09 (Toth et al., 2010). K97/09 is a carbapenemase-expressing strain (blaKPC-2) that is extensively drug-resistant, including ceftazidime, ceftriaxone, imipenem, meropenem, ciprofloxacin, gentamicin and colistin. The A. baumannii strain (ATCC BAA-1605) originated from the tracheal aspirate of a Canadian soldier with ventilator-associated pneumonia. The strain is resistant to ceftazidime, gentamicin, piperacillin, aztreonam, cefepime, ciprofloxacin, imipenem and meropenem (Tien et al., 2007). The E. coli UNT167-1 is a carbapenem resistant strain, isolated from a chronic urinary tract infection case at the University of Texas (Zhanel et al., 2018). The B. pseudomallei strain, 1026b, was originally isolated in 1993 from a 29 year old diabetic rice farmer in Thailand with melioidosis.

PEPTIDES. A3-APO [(H-Chex-Arg-Pro-Asp-Lys-Pro-Arg-Pro-Tyr-Leu-Pro-Arg-Pro-Arg-Pro-Pro-Arg-Pro-Val-Arg)₂-Dab]; Gly11 [(H-Chex-Arg-Pro-Asp-Lys-Pro-Arg-Pro-Tyr-Leu-Gly-Arg-Pro-Arg-Pro-Pro-Arg-Pro-Val-Arg)₂-Dab-NH₂]; Allo-aca, negative control leptin receptor antagonist (Otvos et al., 2011); Gly11, which has the same amino acid sequence as A3-APO except for a change in one residue, fails to bind DnaK and was used to validate DnaK binding as underpinning mechanism of action of A3-APO (Cassone et al., 2008).

### Example

Antibacterial assay. Antibacterial assays are undertaken to determine the minimal inhibitory concentrations (MIC) as described previouslyl⁶. A panel of Gram-negative nosocomial bacteria, E. coli ATCC 29222, K. pneumoniae ATCC 13883, A. baumannii ATCC 19606, and P. aeruginosa ATCC 47085 is chosen to test the antibacterial activities of the peptides described herein, with 2.5×10⁵ cells/ml in MHB at 37° C. immediately prior to the determination of MIC.

### Example

Antibacterial activity. Each C-terminal modified PrAMP DTM-linked tetramer is assayed against nosocomial Gram-negative bacteria, including E. coli ATCC 29222, K. pneumoniae ATCC 13883, A. baumannii ATCC 19606 and P. aeruginosa ATCC 47085. Illustrative results are shown in Table 4.

TABLE 4
Antibacterial activity, MIC (μM), of PrAMPs with different
linkages against Gram-negative nosocomial pathogens.

| Peptide | E. coli | K. pneumoniae | A. baumannii | P. aeruginosa |
|---|---|---|---|---|
| dimer (A3APO) | 2.58 ± 1.14 | 1.44 ± 0.12 | 8.00 ± 1.09 | 3.13 ± 0.05 |
| dimer-NHNH₂ | 2.99 ± 1.00 | 3.63 ± 0.02 | 5.31 ± 0.73 | 3.01 ± 0.18 |
| tetramer (disulfide) | 5.76 ± 1.30 | 3.46 ± 0.33 | 15.80 ± 1.51 | 3.61 ± 1.46 |
| tetramer-DTM-ol | 3.67 ± 0.25 | 2.20 ± 0.01 | 11.61 ± 1.44 | 1.86 ± 0.07 |
| tetramer-DTM-NH₂ | 2.96 ± 0.99 | 2.33 ± 0.01 | 13.29 ± 0.10 | 2.05 ± 0.08 |
| tetramer-DTM-NHNH₂ | 0.93 ± 0.02 | 0.49 ± 0.01 | 2.33 ± 0.48 | 1.14 ± 0.03 |
| tetramer-butene-NHNH₂ | 0.94 ± 0.04 | 0.81 ± 0.02 | 1.89 ± 0.63 | 0.77 ± 0.02 |
| tetramer-bismal-NHNH₂ | 3.93 ± 0.04 | 3.18 ± 0.03 | 10.35 ± 0.22 | 1.62 ± 0.11 |
| tetramer-xylene-NHNH₂ | 6.24 ± 0.53 | 4.49 ± 0.56 | 12.70 ± 0.96 | 1.51 ± 0.33 |

All the tetramers, including tetramer-DTM-NH2, tetramer-DTM-ol, and tetramer-DTM-NHNH₂, maintained a similar activity against E. coli (MIC of 0.93-3.67 μM) and K. pneumoniae (MIC 0.49-2.33 The tetramers increasingly improved the potency against A. baumannii (MIC 2.33-13.29 μM) and P. aeruginosa (MIC 1.14-2.05 which are strongly associated with hospital associated/acquired infections²⁹. Among the tetramers, the tetramer-DTM-NHNH₂ showed surprisingly high activity against all of the Gram-negative nosocomial bacteria with MIC=0.49-2.33 which was significantly improved compared with the disulfide-linked tetramer and dimer (A3-APO) hydrazides. These results highlight the significant advantage conferred to bacterial killing by increased multimerization together with C-terminal peptide hydrazide modification. The chemical basis for the improvement afforded by the presence of the C-terminal hydrazide of Chex1-Arg20 in both bacterial selectivity and potency remains unknown. Hydrazide-reactive peptides have been used to label bacterial cell lysates³⁰, and may possibly react more strongly with proteins.

Although the DTM tether has been demonstrated to be reducible by glutathione leading to release of the dansylthiol as reported for a DTM moiety-linked protein³¹, the rate of cleavage is slower than of the disulfide bond that was used in the previous generation of tetrameric Chex1-Arg20. The alternative functional linkers examined here are each expected to have similar strong or complete stability against the reductive conditions within cells. The resulting tetrameric hydrazide PrAMPs (Scheme 2) were also each tested on a panel of nosocomial Gram-negative bacteria. The MIC values also showed significantly improved antibacterial activities for all the peptides with the tetramer-NHNH₂ bearing the shortest linker (butene) showing an MIC of 0.77 μM-1.89 μM against E. coli, K. pneumoniae, A. baumannii and P. aeruginosa (Table). The two other tetramers-NHNH₂, linked via bismaleimide or xylene, had similar activities (MIC 1.51 μM-6.24 μM) against E. coli, K. pneumoniae and P. aeruginosa, but not against A. baumannii (MIC 10.35 μM-12.70 Especially noteworthy are the pronounced actions of the tetrameric peptides against P. aeruginosa. This bacteria is reportedly more difficult to treat than most, if not all, other Gram negative bacteria and the observed MIC values in general 4-8-fold higher than for other nosocomial pathogens. For example, the tetramer-butene-NHNH₂ possesses an MIC of just 0.77 μM.

### Example

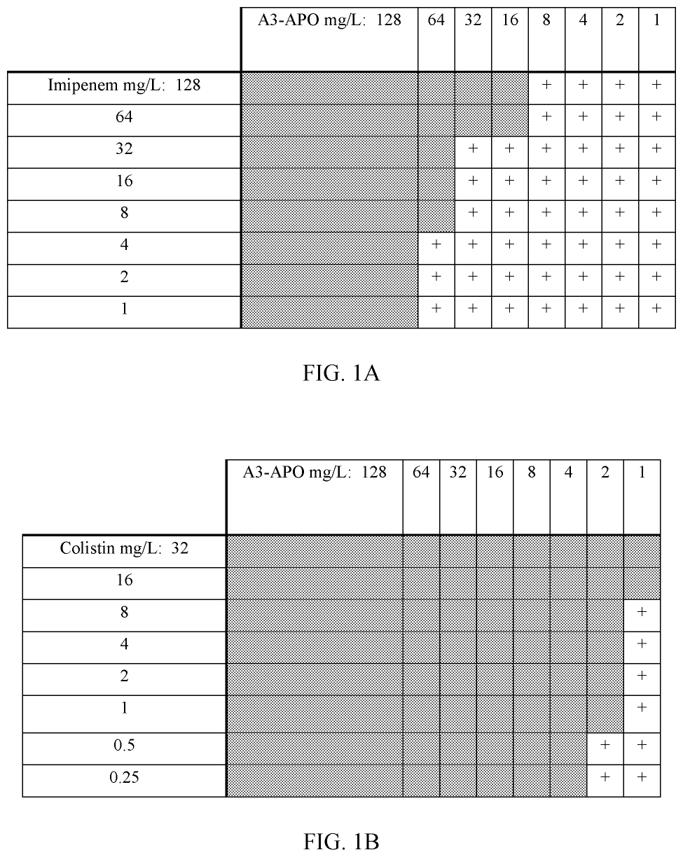

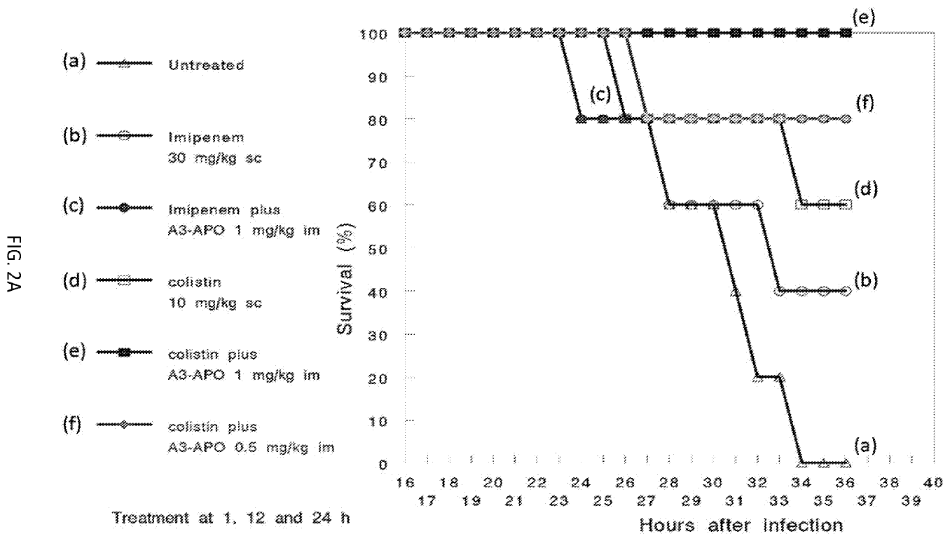

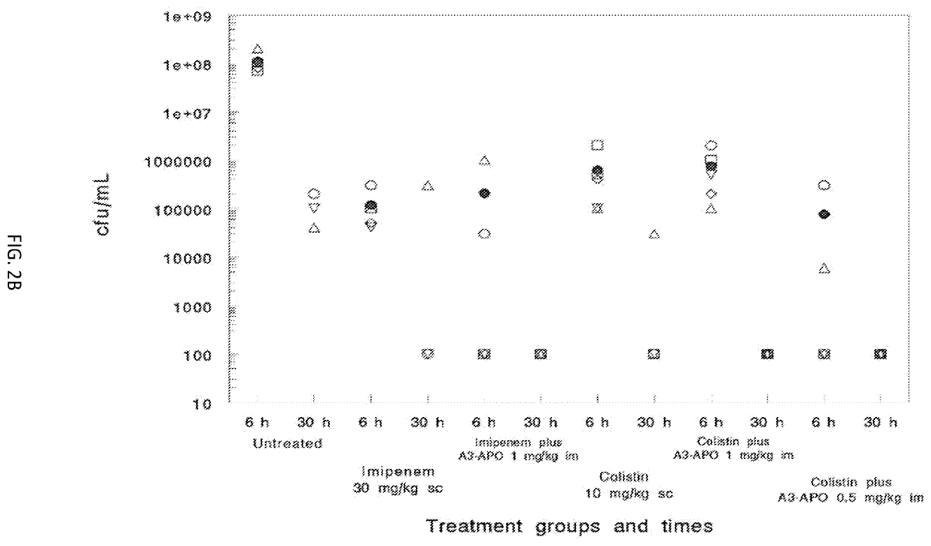

Restoring antibiotic sensitivity to resistant bacteria. A colistin-resistant, carbapenem-resistant strain of K. pneumonia (K97/09) from a clinical isolate was used to test the effect of A3-APO treatment on antibiotic resistance. An in vitro assay measuring visible bacterial growth in the presence of A3-APO and imipenem or colistin was performed. The results are shown in FIGS. 1A-1B, demonstrating synergy between A3-APO and both antibiotics. Additional experimental results showing synergy are shown in the following table.

Synergy Results for Co-administration of A3-APO (ARV-1501)

| Co-administered | | |
|---|---|---|
| Antibiotic | Pathogen | Outcome |
| amoxicillin | E. coli | reversal of TEM-1 related |
| | | resistance |
| amoxicillin | H. influenzae | MIC lowered with priming |
| chloramphenicol | H. influenzae | MIC lowered with priming |
| chloramphenicol | K. pneumoniae | MIC lowered with priming |
| chloramphenicol | E. coli | MIC lowered with priming |
| colistin | K. pneumoniae | MIC decrease from >64 to 1 |
| sulfamethoxazole | S. typhimurium | MIC lowered with priming |
| trimethoprim/ | E. coli | MIC lowered with priming |
| sulfamethoxazole | | |

### Example

In vitro activity and synergy. Minimal inhibitory concentration (MIC) assays were performed using sterile 96-well polypropylene plates in a final volume of 100 mL. Briefly, 50 μL of midlogarithmic phase bacterial cultures were diluted to 5×105 CFU/mL in Mueller-Hinton broth (MHB) and then added to 50 μL of the serially diluted antibiotic. The highest A3-APO and other test compound concentration evaluated was 256 mg/L. Cultures were incubated at 37° C. for 16-20 h without shaking. MICs were identified as the lowest antimicrobial concentrations at which turbidity was not observed. Antimicrobial synergy was determined by evaluating the fractional inhibitory concentration (FIC) index and was characterized by a conventional checkerboard assay (Fernandez-Cuenca et al., 2003). Bacteria grown to mid-logarithmic phase in MHB were pre-incubated with serially diluted concentrations of peptides such as A3-APO, and the antimicrobial controls, imipenem, colistin or meropenem.

The sum of the FICs (Σ FIC) was calculated with the equation Σ FIC=FICA+FICB=(CA/MICA)+(CB/MICB), where MICA and MICB are the MICs of antimicrobial A and B alone, respectively, and CA and CB are the concentrations of the drugs when combined, respectively. Synergy was defined as a Σ FICs≤0.5 and additive activity was defined as a Σ FICs>0.5≤1.0.

### Example

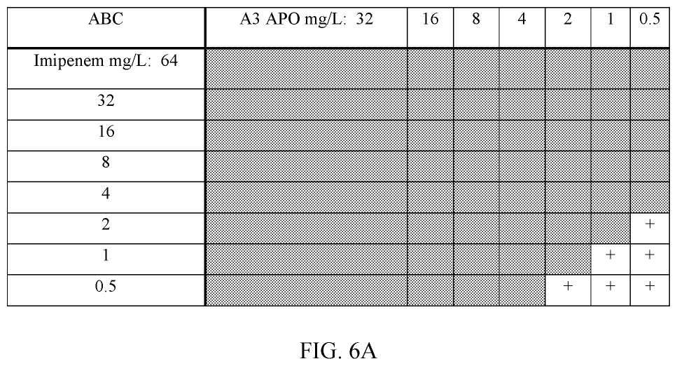

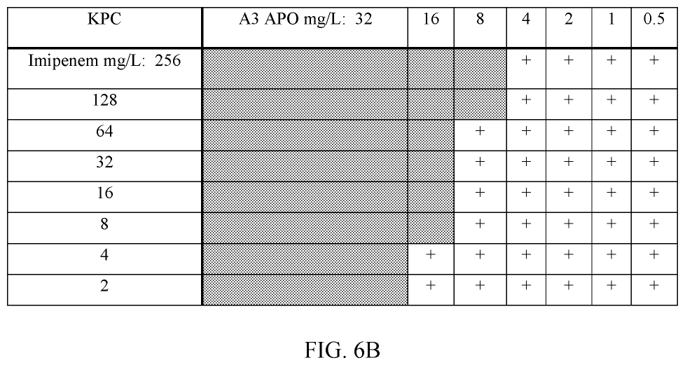

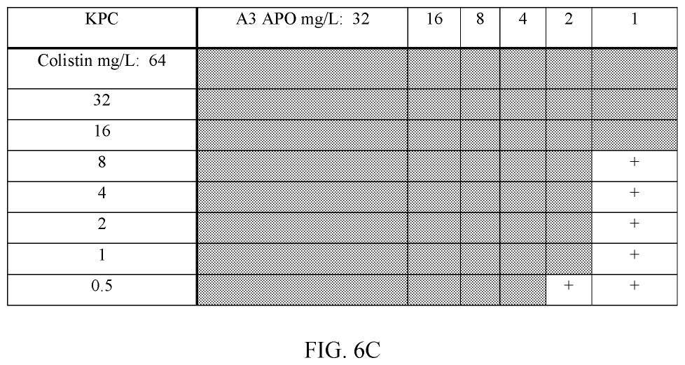

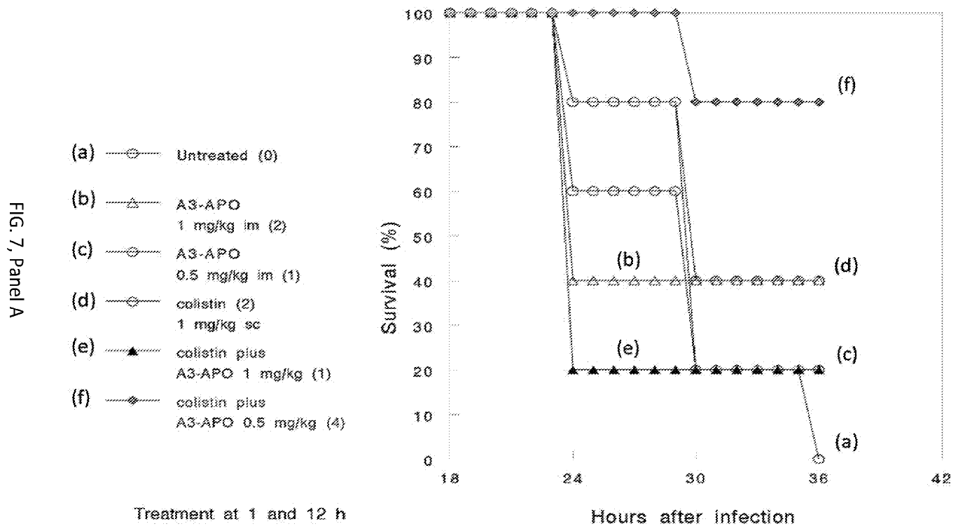

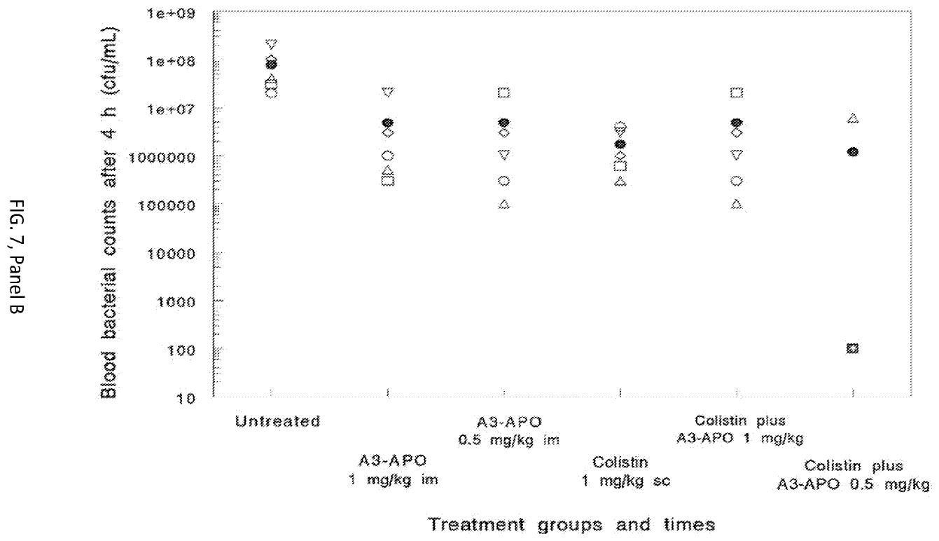

In vitro activity and synergy. MIC values of A3-APO, colistin, and imipenem against the K. pneumoniae strain (K97/09) were 32 mg/L, 64 mg/L, and >256 mg/L, respectively. MIC values of A3-APO, colistin and imipenem against the A. baumannii strain (BAA-1605) were 32 mg/L, <0.5 mg/L and 64 mg/L, respectively. Combining antimicrobials against K97/09 resulted in strong synergy for the A3-APO/colistin combination (ΣFIC=0.08, FIG. 6 top panel) and borderline synergy or additive activity for the A3-APO/imipenem combination (ΣFIC=0.53, FIG. 6 second panel). Combining imipenem and A3-APO against BAA-1605 (colistin is not evaluated because BAA-1605 is a colistin-sensitive strain) resulted in strong synergy for the A3-APO/imipenem combination (ΣFIC=0.08, FIG. 6 third panel). The negative control Allo-aca peptide or peptide Gly11, an A3-APO analog that fails to bind bacterial DnaK (Cassone et al., 2008), had no activity on either pathogen (MICs>256 mg/L), and failed to exert any improvement in the MIC values when added together with either imipenem or colistin suggesting that the effect is specific and can be correlated with DnaK inhibition resulting in inhibition of resistance enzymes.

### Example

In vivo Studies. Animals. Assays 1-2. NMRI (Naval Medical Research Institute) BR or CD-1 mice (Toxi-Coop Zrt, Budapest, Hungary) were housed in plastic type 2 cages, 3-5 mice per cage, on softwood granules as bedding. The room was kept between 21° C. and 25° C. with 12 h light:12 h dark cycles. The animals had free access to tap water and pelleted rodent food. Upon completion of the experiments, surviving mice were euthanized by diethyl ether inhalation. Animals were maintained and handled in accordance with the recommendations of the Guidelines for the Care and Use of Laboratory Animals, and the protocols were approved by the Animal Care Committee of Semmelweis University. The planned 15 treatment groups of mice were divided roughly equally into two assays with 8 and 7 treatment groups and untreated controls in each assay for safe and humane handling of large numbers of mice.

Infection models. NMRI mice weighing approximately 20 g (4 weeks old) were infected by intraperitoneal (ip) injection of 4×108 CFU/g K. pneumoniae K97/09. Mice were randomly allocated to 8 and 9 groups (5 mice per group).

Bacteremia Synergy Assay 1.

Group 1: phosphate buffered saline (PBS) subcutaneously (sc) 1 h after infection.
Group 2: imipenem 30 mg/kg sc at 2, 14 and 26 h after infection.
Group 3: A3-APO 1 mg/kg im 1, 13 and 25 h after infection, imipenem 30 mg/kg sc at 2, 14 and 26 h after infection.
Group 4: colistin 10 mg/kg sc at 2, 14 and 26 h after infection.
Group 5: A3-APO 1 mg/kg im 1, 13 and 25 h after infection, colistin 10 mg/kg sc at 2, 14 and 26 h after infection.
Group 6: A3-APO 0.5 mg/kg im 1, 13 and 25 h after infection, colistin 10 mg/kg sc at 2, 14 and 26 h after infection.
Group 7: A3-APO 1 mg/kg im 1, 13 and 25 h after infection, colistin 1 mg/kg sc at 2, 14 and 26 h after infection.
Group 8: A3-APO 0.5 mg/kg im 1, 13 and 25 h after infection, colistin 1 mg/kg sc at 2, 14 and 26 h after infection.
Group 9: colistin 10 mg/kg sc 2 h after infection, A3-APO 1 mg/kg im 5 h after infection.

Survival was recorded hourly 24-36 h after infection. Blood samples (10 μL) were taken from the tail vein to determine the bacterial burden at 6 and 30 h after infection from all surviving animals. Groups with 2 or more animal having blood bacterial counts below the level of detection (1×103 CFU/mL) at 6 h post-infection were excluded from analysis due to presumption of low inoculum or rapid host clearance. The blood was prevented from coagulation with EDTA and the samples were serially diluted in 0.9% saline. Each dilution was cultured providing a detectable threshold of 10³ CFU/mL.

Bacteremia SYNERGY ASSAY 2.

Group 1: PBS sc 1 h after infection.
Group 2: Colistin 1 mg/kg sc at 2 and 13 h after infection.
Group 3: A3-APO 1 mg/kg im 1 and 12 h after infection.
Group 4: A3-APO 1 mg/kg im 1 and 12 h after infection, colistin 1 mg/kg sc at 2 and 13 h after infection.
Group 5: Colistin 10 mg/kg sc 4 h after infection, A3-APO 1 mg/kg im 6 h after infection.
Group 6: A3-APO 0.5 mg/kg im 1 and 12 h after infection.
Group 7: A3-APO 0.5 mg/kg ip 1 and 12 h after infection.
Group 8: A3-APO 0.5 mg/kg im 1 and 12 h after infection, colistin 1 mg/kg sc at 2 and 13 h after infection.

Survival was monitored at 12, 24 and 36 h and blood samples were taken 4 and 11 h after infection and worked up as in Assay 1. Blood bacterial load reduction and survival in the various groups were compared with Chisquare and unpaired Student's t-testing, respectively (Microsoft Excel, Microsoft, 2007, Redmond, Wash., USA, and SlideWrite, Encinitas, Calif., USA).

### Example

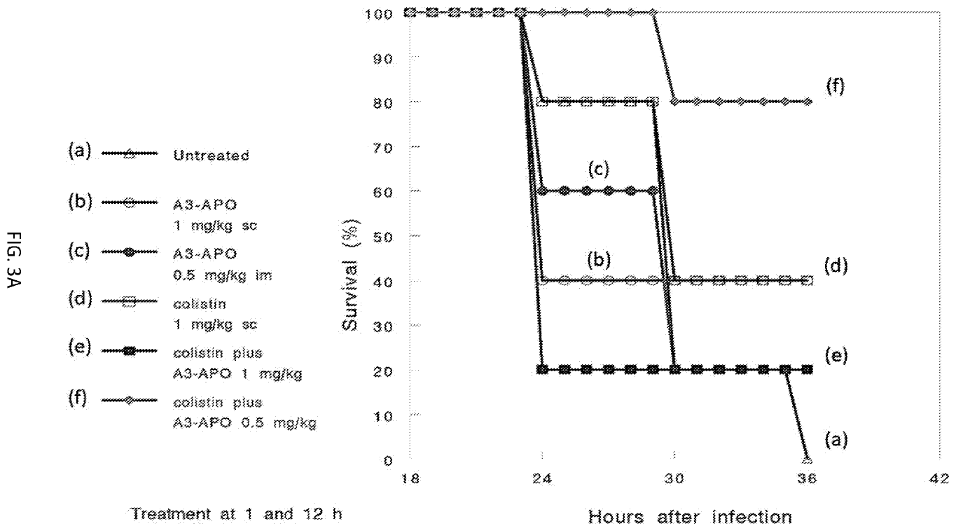

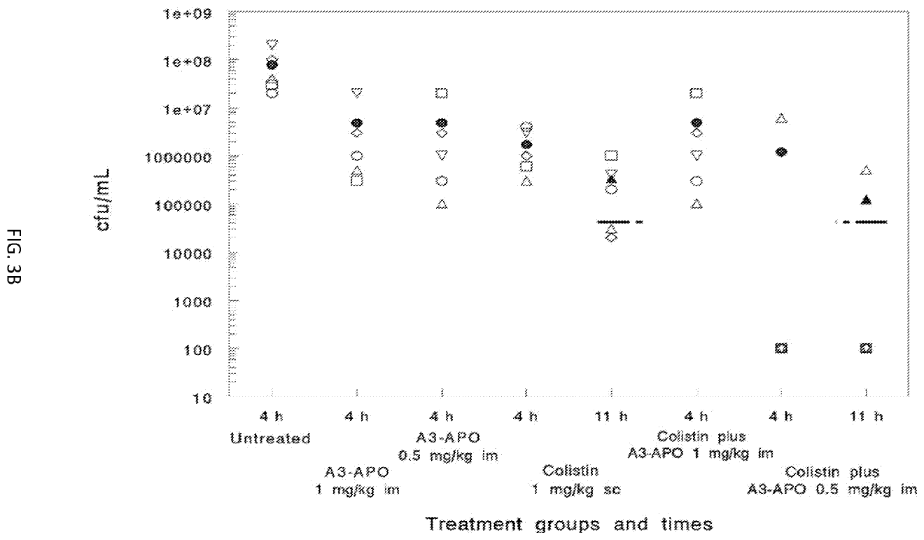

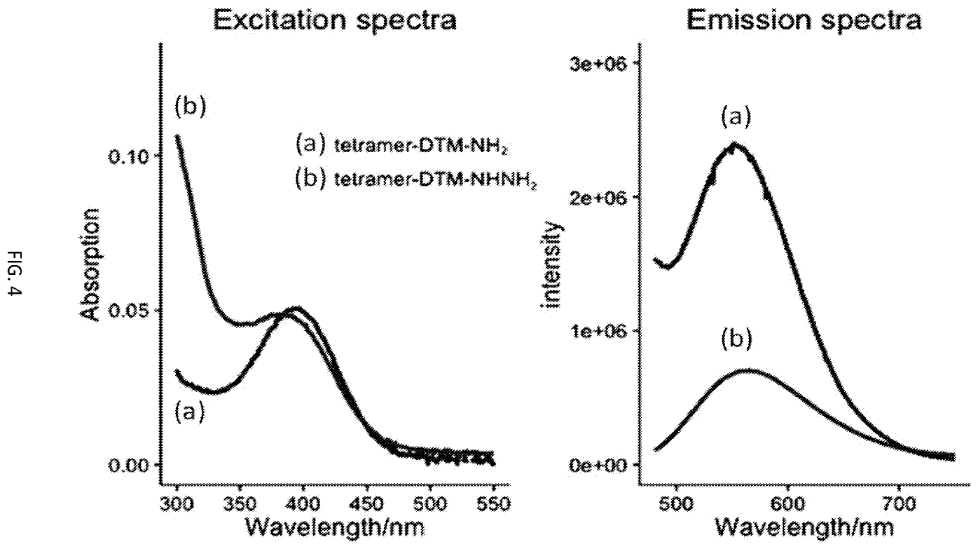

The combination of A3-APO with other antibiotics was tested in a mouse model of systemic Klebsiella pneumoniae infection. The results based on survival and blood bacterial counts are shown in FIGS. 2A-2B. The combination of A3-APO significantly reduced doses of colistin tested alone in the same mouse model. The results based on survival and blood bacterial counts are shown in FIGS. 3A-3B.

TABLE 2
Antimicrobial combination chemotherapy results.

| | | Assay 1 | Assay 1 |
|---|---|---|---|
| Antibiotic combination | Assay 1 Treatment | Survival % | cfu/mL |
| Colistin 1 mg/kg + | A3-APO 1, 13, 25 h | 100 | <1000 |
| A3-APO 0.5 mg/kg | Colistin 2, 14, 26 h | | at 6 h |
| Colistin 10 mg/kg + | Colistin 2 h | 100 | <1000 |
| A3-APO 1 mg/kg | A3-APO 5 h | | at 6 h |

| | | Assay 2 | Assay 2 |
|---|---|---|---|
| Antibiotic combination | Assay 2 Treatment | Survival % | cfu/mL |
| Colistin 1 mg/kg + | A3-APO 1, 12 h | 80 | 1.2 × 10⁶ |
| A3-APO 0.5 mg/kg | Colistin 2, 13 h | | at 4 h |
| Colistin 10 mg/kg + | Colistin 4 h | 0 | 1.3 × 10⁸ |
| A3-APO 1 mg/kg | A3-APO 6 h | | at 4 h |

The 36 h survival data and the mean blood bacterial counts are calculated from 5 mice per group. Note that in Assay 2 of the treatment failure recovery group (colistin 10 mg/kg plus A3-APO 1 mg/kg late after infection) the bacterial load represents untreated animals (the single treatment was administered after blood sampling). It is noteworthy that the co-administration described herein is successful even when dosing b.i.d.

### Example

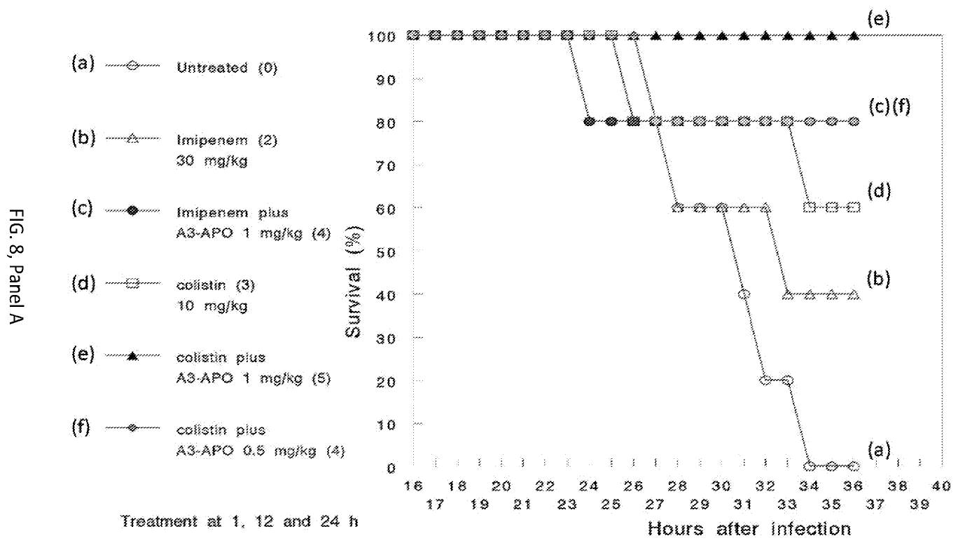

The addition of A3-APO to colistin prolongs survival when compared to placebo. It has been reported that a single agent therapeutic dose of A3-APO in a murine bacteremia infection model is about 5 mg/kg im (Ostorhazi et al., 2011a). It has also reported that the activity, as monotherapy, of A3-APO, demonstrated a dose-dependent survival benefit (Szabo et al., 2010). It has been discovered that lower doses than 5 mg/kg may be efficacious when used in conjunction with either colistin or imipenem in a K. pneumoniae bacteremia infection model. When given as monotherapy, either 0.5 mg/kg or 1.0 mg/kg im (Groups 6 and 3 in Assay 2) resulted in a survival advantage of 20-40%; also identified was an improvement in blood bacterial count reduction compared to untreated animals (FIGS. 7A and 7B). When administered ip, a dose of 0.5 mg/kg appeared to be less efficacious than the same dose administered im (Group 7 in Assay 2, data not shown).

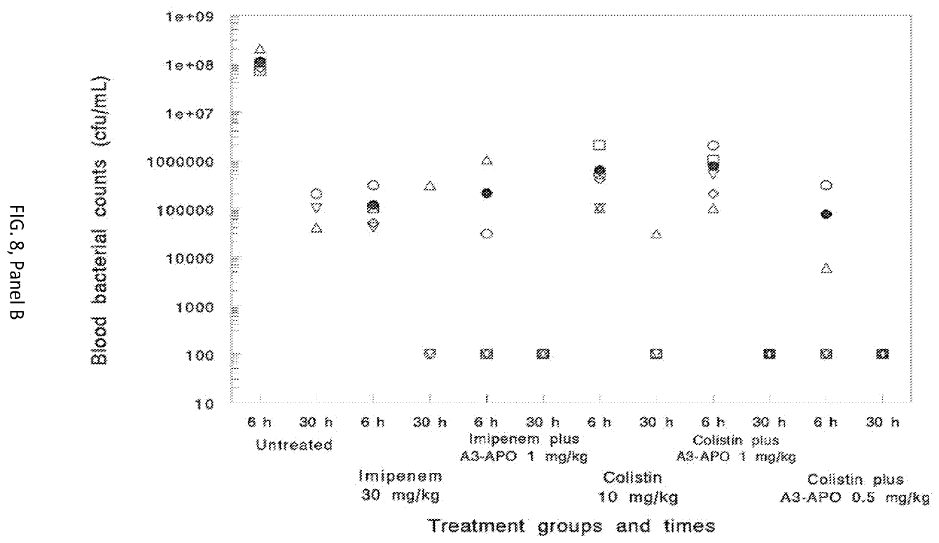

Assay 1—Imipenem and Colistin. Imipenem administered 3 times sc at 30 mg/kg (Group 2) demonstrated a 40% survival improvement at 36 h over untreated controls (Group 1) (FIG. 8A) or the 30-h blood bacterial counts (FIG. 8B). Colistin administered at 10 mg/kg sc (Group 4) was more effective (60% survival) but had a lower blood CFU reduction (FIGS. 8A and 8B). When used in combination with 1 mg/kg A3-APO administered im 1 h prior to antimicrobial administration, a significant improvement in the 36-h survival rate was noted (80% with imipenem, Group 3, and 100% with colistin, Group 5) together with sterilization of the blood of the surviving animals at 30 h after infection (FIGS. 8A and 8B). A3-APO improved the survival rate even at the lowest dose evaluated (0.5 mg/kg im) when administered with 10 mg/kg colistin (Group 6, 80% at 36 h, FIG. 8A). Comparing use of either antimicrobial with A3-APO to monotherapy arms alone, shows a surprising improvement in survival (hazard ratio=0.70, 95% CI=±0.45; p<0.005). The combination therapy does not appear to be associated with organ toxicity. After necropsy, the weights of the heart, kidney, spleen and liver exhibited no deviation from those of untreated control animals, or among treated groups (data not shown).

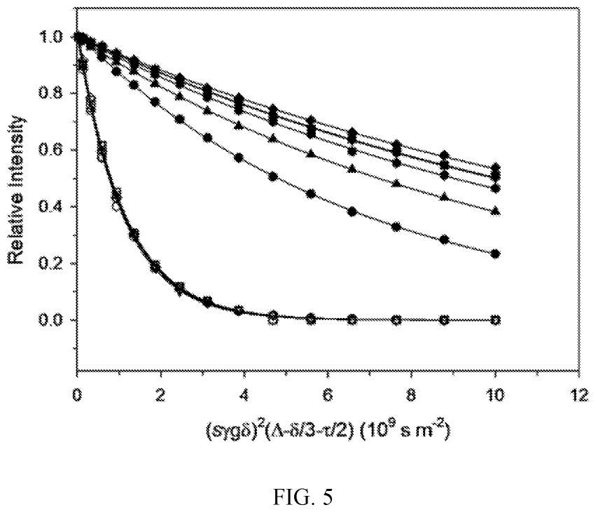

Assay 2—Reduced Colistin Dosing. A tenfold reduction in the colistin dose (Group 2) and lowering the frequency from thrice to twice resulted in poor comparative survival at 36 h (40%) and even less impact at reducing the bacterial burden early at the assay course (<1 log 10 unit after 6 h, FIGS. 7A and 5B). Surprisingly, a combination of a subtherapeutic dose of colistin and a low dose of 0.5 mg/kg A3-APO (Group 8) improved survival (80% survival, FIG. 7A). The lower dose of A3-APO (0.5 mg/kg) appears to have a greater effect on survival than the higher dose of A3-APO at 1.0 mg/kg, though the statistical significance of those two results is has not been determined. When 1 mg/kg colistin was combined with 0.5 mg/kg A3-APO under the conditions of Assay 1 (three antibiotic doses, Group 8), 100% survival was observed with bacterial counts of each mice below the 10³ CFU/mL detection limit throughout the course of the experiment (data not shown).

### Example

Dose-dependent and time-dependent efficacy of peptides. CD-1 mice of 8 weeks are infected ip with 6.8×10⁸ CFU/g of the extended spectrum β-lactamase producing E. coli 5770 strain (Szabo et al., 2010). Test compound is administered ip at a 2.5, 5 and 10 mg/kg dose at 4, 8 and 12 h post-infection. Prior to drug administration at all timepoints and 4 h and 20 h later (16 h and 24 h post-infection), 10 μL blood was taken from the tail vein of 3 mice for determining blood bacterial counts.

### Example

Melioidosis model. Mean inhaled doses of 58×LD50 (2 separate sprays of 56 and 60 LDs) of Burkholderia pseudomallei 1026b were administered to 6-8 week-old female Balb/c mice by whole-body aerosol. Aerosol was generated using a three-jet collision nebulizer. All aerosol procedures were controlled and monitored using the Automated Bioaerosol Exposure system (Hartings and Roy, 2004) operating with a whole-body rodent exposure chamber. Integrated air samples were obtained from the chamber during each exposure using an all-glass impinger. Mice were randomly placed into separate cages upon the conclusion of each aerosol. Cohort size for statistical evaluation was 10 mice. Ceftazidime was administered ip at 300 (Group 1) or 150 mg/kg (Group 2) doses beginning 24 h post-challenge four times a day and treatment continued for 21 days. Three additional groups receiving 150 mg/kg ceftazidime ip were treated simultaneously with 2.5, 5 or 10 mg/kg test compound added im (Groups 3-5). A vehicle control group received 0.2 mL saline sc four times a day. Survival was monitored twice daily during treatment and once daily thereafter. Moribund animals were euthanized as necessary and counted as dead. In accordance with the protocol approved by the Institutional Animal Care and Use Committee of the United States Army Medical Research Institute of Infectious Diseases, the study was terminated at day 62. At the conclusion of the study all animals were humanely euthanized and target organs (spleens and lungs) were harvested for the determination of bacterial loads. The results were processed with a stratified Kaplan-Meyer analysis with a logrank test as implemented on Prism Version 5.04 GraphPad.

### Example

Cytotoxicity. In vitro cytotoxicity was also determined via the Promega CellTiter 96 Aqueous Non-Radioactive Cell Proliferation Assay¹⁴ using the mammalian cell lines, HEK-293 (ATCC CRL 1573) and H-4-II-E (ATCC CRL-1548). All of the peptides evaluated did not show toxicity on either mammal cell lines at the highest tested concentration (25 μM), which is much higher than the antibacterial activity MIC (Table 1) on nosocomial Gram-negative bacteria.

### Example

Cell proliferation test. The proliferation of HEK-293 (ATCC® CRL-1573™) and H-4-II-E (ATCC® CRL-1548™) cells were tested with tetrameric PrAMPs with CellTiter 96 AQᵤₑₒᵤₛ Non-Radioactive Cell Proliferation Assay (Promega) as described before¹⁶, which was measured at 490 nm.

### Compound Examples

Materials. 9-Fluoroenylmethoxylcarbonyl (Fmoc)-L-amino acids, 2-(6-chloro-1H-benzotriazole-1-yl)-1,1,3,3-tetramethylamonium hexafluorophosphate (HCTU), and 2-chlorotrityl chloride resin are purchased from GL Biochem (Shanghai, China). TentaGel-MB-RAM-resin is from Rapp Polymere (Tubingen, Germany). N,N-Diisopropylethylamine (DIPEA), dimethylformamide (DMF), and trifluoroacetic acid (TFA) are obtained from Auspep (Melbourne, Australia). 1,6-Bimaleimidohexane is obtained from TCL (Gillman, Australia). Isobutyl chloroformate (IBCF), NaBH4, ethyl acetate, 2,3-dibromomaleimide, α,α′-dibromo-p-xylene, trans-1,4-dibromo-2-butene, piperidine, triisopropylsilane (TIPS), anisole, 3,6-dioxa-1,8-octanedithiol (DODT), hydrazine monohydrate, and acetonitrile (CH₃CN) are all obtained from Sigma (Sydney, Australia).

### Example

Peptide preparation. The peptides are synthesized by Fmoc/tBu solid-phase methods²⁴. Dimeric peptide synthesis is carried out on a CEM Liberty microwave-assisted synthesizer using TentaGel-MB-RAM-resin or 2-chlorotrityl chloride resin as previously described¹⁴ (Scheme 1). The C-terminal modified peptides are prepared on 2-chlorotrityl chloride resin functionalized with hydrazide or N-Fmoc-amino acid alcohol prior to SPPS. Standard Fmoc-chemistry is used throughout with a 4-fold molar excess of the Fmoc-protected amino acids in the presence of 4-fold HCTU and 8-fold DIPEA. The peptides are cleaved from the solid support resin with TFA in the presence of anisole, TIPS and DODT as scavenger (ratio 95:2:2:1) for 2 h at room temperature. After filtration to remove the resin, the filtrate is concentrated under a stream of nitrogen, and the peptide products are precipitated in ice-cold diethyl ether and washed three times. The peptides are then purified by reversed-phase high performance liquid chromatography (RP-HPLC) in water and acetonitrile containing 0.1% TFA. The final products are characterized by both RP-HPLC and matrix-assisted laser desorption/ionization time-of-flight mass spectrometry (MALDI-TOF MS).

### Example

Preparation of different length linked tetramer-hydrazides. To a solution of dimeric PrAMP-hydrazide (0.5 mmol) in guanidine hydrochloride buffer (pH 8.0, 150 μL) is added dropwise a solution of linker including (DTM, trans-1,4-dibromo-2-butene, 1,6-bismaleimidohexane, or α,α′-dibromo-p-xylene) that is dissolved in acetonitrile (0.25 mmol, 100 The mixture is reacted for 2-6 h at room temperature and the resulting tethered tetrameric peptides are purified by RP-HPLC in aqueous acetonitrile containing 0.1% TFA in overall moderate yield (Scheme 2). The final products are characterized by RP-HPLC and MALDI-TOF MS.

### Example

Homotetramers with C-terminal modification of PrAMPs. Dimeric Chex1-Arg20 peptides with certain C-terminal modifications, such as the hydrazide, alcohol, amide, and the like of Chex1-Arg20, are prepared via Fmoc/tBu solid phase peptide synthesis (SPPS) with different functionalized 2-trityl chloride resins which are prepared in presence of hydrazine (2 equiv) or N-Fmoc-S-trityl-L-cysteine-β-amino-alcohol (Scheme 1Scheme 1). Each C-terminal modified dimer bearing a single C-terminal region Cys residue is then subjected to reaction in solution with a dibromomaleimide moiety under mild conditions (guanidine hydrochloride buffer, pH 8.0) to produce three dithiolmaleimide-linked tetramers (Scheme 2). Tetrameric PrAMP-hydrazide is also prepared using either trans-1,4-dibromo-2-butene, 1,6-bimaleimidohexane, or α,α′-dibromo-p-xylene as tether (Scheme 2) to investigate the effect of length on antibacterial activity. Each tetramer is obtained in overall yield of ca. ˜15% relative to crude Chex1-Arg20 dimer and then subjected to comprehensive chemical characterization including analytical RP-HPLC and MALDI-TOF MS to confirm their purity.

### Example

Photoluminescence. Spectra were obtained using a HORIBA Jobin Yvon Fluorolog fluorescence spectrophotometer. To minimise reabsorption effects, the optical absorptions of the sample solutions were kept around 0.10 at the excitation wavelength. The sample solutions of tetramer-DTM-NH₂ or tetramer-DTM-NHNH₂ were prepared in MilliQ water in BRAND® disposable cuvettes and spectra were recorded immediately after the UV-vis absorbance measurements.

### Example

Photoluminescence analysis. The photoluminescent properties of the DTM-linked homotetramers were determined and showed a λ_{ex,max} of 492-495 nm and λ_{em,max} of 554-555 nm (FIG. 4). The excitation and emission wavelength of DTM fluorescence matched with the Qdot® 565 probe (Thermo Fisher Scientific) for potential imaging and flow cytometry applications. These properties augur well for the use of these tetrameric peptides in their use in, for example, mechanism studies involving high resolution microscopy and are an alternative to the bulky AlexFluor 647 or 430 that can cause loss of antibacterial activity^{14, 23}.

### Example

Diffusion NMR spectroscopy. Translational diffusion coefficients of PrAMPs were measured at 298 K on a Bruker Avance II 800 MHz spectrometer using a TXI cryoprobe equipped with a single gradient (Gz). Diffusion measurements were carried out using a standard BPP-STE sequence without modification (stebpgp1s, Bruker pulse sequence library). The field gradient strength of Gz was calibrated by measuring the self-diffusion coefficient of residual H₂O in a 100% ²H₂O sample at 298.13 K²⁵ using a diffusion coefficient of 1.9×10⁻⁹ m² s⁻¹ for the residual H₂O²⁶. Spectra were processed in TOPSPIN (Version 3.2, Bruker). Diffusion coefficient, D, was determined by fitting diffusion weighted intensities of well resolved and intense peaks using the T₁/T₂ relaxation module in TOPSPIN (Version 3.2, Bruker) and SigmaPlot (version 12.5, Systat Software) to the following equation:

I=I₀ exp{−γ²s²g²δ²(Δ−δ/3−τ/2)D} (1)

where γ is the gyromagnetic ratio of protons and s, g, δ, and Δ are the shape factor, amplitude, duration and separation, respectively, of the single pair of gradient pulses, and τ being the time interval within the bipolar pulse pair. Sinusoidal shaped gradient pulses were used in the present study. The effective hydrodynamic radius of PrAMPs were subsequently estimated from experimentally measured translational diffusion coefficients using the following relationship²⁷:

Rₕ=Rₕ^{REF}(D^{REF}/D) (2)

where Rₕ^{REF} and D^{REF} are the hydrodynamic radius and translational diffusion coefficient, respectively, of the reference molecule. Dioxane with Rₕ^{REF} of 2.12 Å is illustratively used as the reference molecule²⁸.

### Example

Diffusion NMR. To further delineate the potential relationship between the size/shape of these peptides with different chemical tethers in isotropic solution and their activity against pathogens, translational diffusion coefficients of PrAMPs were measured using PFG-NMR and the results are summarized in Table 3.

TABLE 1
NMR diffusion coefficients of PrAMPs
with C-terminal hydrazide*.

| | | D^{p} | D^{d} | |
|---|---|---|---|---|
| | C | (×10⁻¹⁰ | (×10⁻¹⁰ | Rₕ |
| Peptide | (□M) | m² s⁻¹) | m² s⁻¹) | (Å) |
| dimer-NHNH₂ | 61 | 0.956 ± 0.004 | 9.052 ± 0.087 | 20.07 ± 0.21 |
| tetramer-DTM- | 17 | 0.767 ± 0.004 | 9.391 ± 0.177 | 25.96 ± 0.51 |
| NHNH₂ | | | | |
| tetramer-butene- | 31 | 0.694 ± 0.002 | 8.814 ± 0.111 | 26.92 ± 0.35 |
| NHNH₂ | | | | |
| tetramer-bismal- | 26 | 0.688 ± 0.004 | 8.807 ± 0.038 | 27.14 ± 0.20 |
| NHNH₂ | | | | |
| tetramer-xylene- | 43 | 0.626 ± 0.003 | 9.130 ± 0.104 | 30.92 ± 0.38 |
| NHNH₂ | | | | |

*C is the concentration of the PrAMPs used for the diffusion NMR measurements, D^{p} refers to the diffusion coefficient of PrAMPs in deuterated water, D^{d} to the diffusion coefficient of dioxane in deuterated water, and Rₕ is the effective hydrodynamic radius of the PrAMPs.

Based on the well-known Stokes-Einstein equation, Dₜ=k_{B}T/(6πηRₕ), the molecules with larger hydrodynamic radius Rₕ would display a slower diffusion coefficient D in solution.

The dimer-NHNH₂ exhibited a 34.6% reduction (Table 1) in translational diffusion coefficient, which is in excellent agreement with a reduction of 33.3% as predicted by the Stokes-Einstein equation for spheres. In contrast, the tetramer-hydrazides displayed a further reduction in their diffusion coefficients ranging from 19.8% to 34.6% (Table 1) when compared with dimeric hydrazide. Such range in reduction suggests no aggregation or oligomeric formation of the tetrameric PrAMPs.

Amongst the four different linked tetramer-hydrazides, tetramer-bismal-NHNH₂ and tetramer-xylene-NHNH₂ displayed an increased hydrodynamic radius by ˜35% and −54%, respectively, relative to dimer-NHNH₂ (Table 1). Such increase indicated that the tetramer-DTM-NHNH₂ and tetramer-butene-NHNH₂ were more compact which may correlate with the observed increased activity against Gram-negative bacteria. The peptide chains may possibly act synergistically against the membrane in a similar manner to PGLa/magainin against E. coli lipid extract membrane bilayers³², where PGLa inserts across the bilayer and magainin adopts a surface orientation, and together more severely disrupt the membrane. Possibly some peptide chains could insert into the membrane with others from the tetramer lying on the surface and act in a similar manner to PGLa/magainin.

### Example

Circular dichroism (CD) spectroscopy. As an indication of the secondary structures of these tetramer-hydrazide PrAMPs, circular dichroism (CD) spectroscopy was used. The CD spectra indicated no significant difference in secondary structure among these peptides which were mainly unstructured in 20 mM, phosphate buffer, pH 7.4, as reported for other similar PrAMPs³². Two-dimensional ¹H NMR spectra of dimeric forms of PrAMP also indicated a primarily unstructured conformation in solution, most likely due to the high positive charge on the peptides.

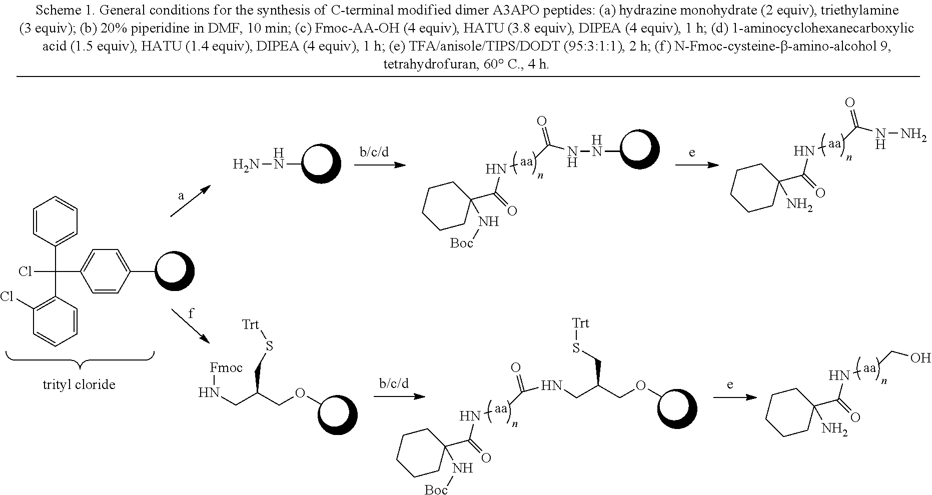

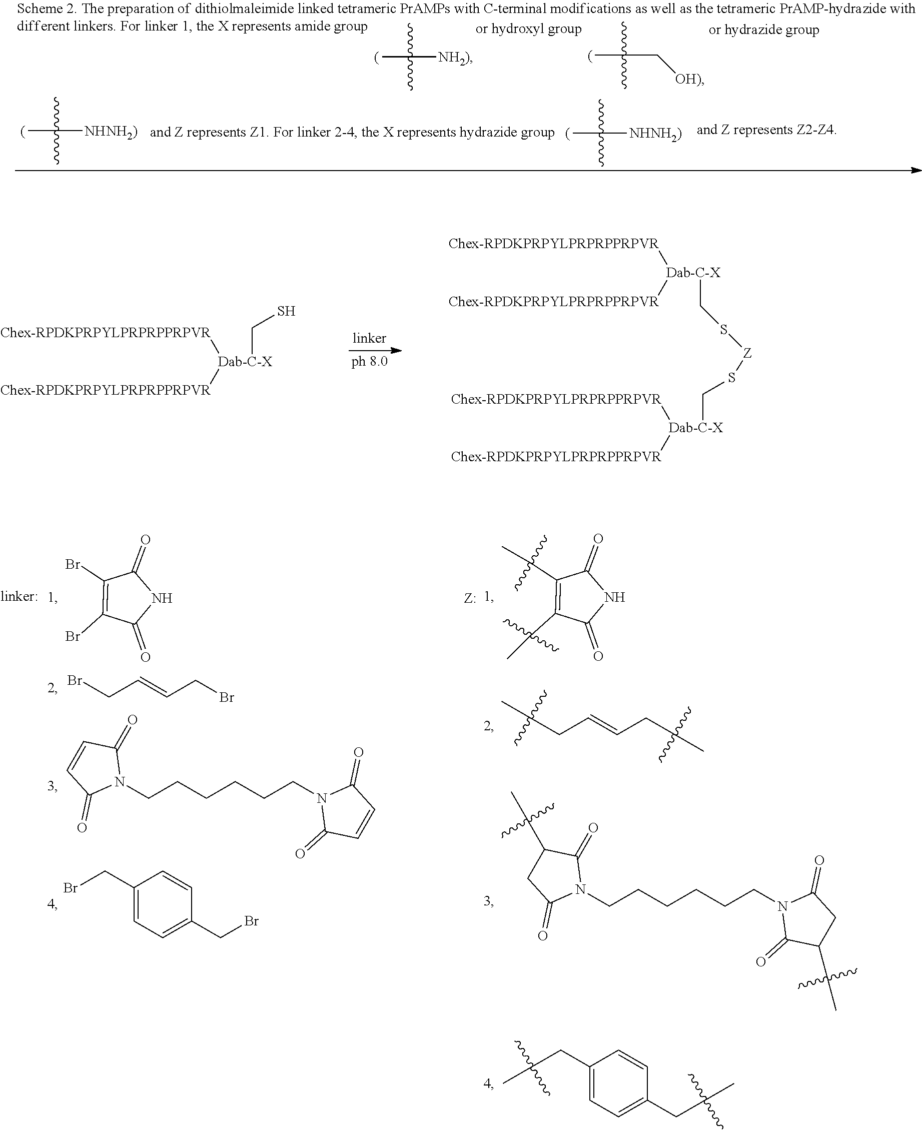

The foregoing is illustrative of the invention described herein, and is not to be construed as limiting thereof. The invention may also be embodied in different forms and should not be construed as limited to the embodiments set forth herein. The invention is also defined by included drawing, and the following claims, with equivalents of the claims to be included therein.

The following publications, and each of the additional publications cited herein are incorporated herein by reference:
1. World Health Organization, 2014.
2. A. L. Hilchie, K. Wuerth and R. E. W. Hancock, Nat. Chem. Biol., 2013, 9, 761-768.
3. L. Otvos, Jr., J. D. Wade, F. Lin, B. A. Condie, J. Hanrieder and R. Hoffmann, J. Med. Chem., 2005, 48, 5349-5359.
4. P. Czihal, D. Knappe, S. Fritsche, M. Zahn, N. Berthold, S. Piantavigna, U. Muller, S. Van Dorpe, N. Herth, A. Binas, G. Kohler, B. De Spiegeleer, L. L. Martin, O. Nolte, N. Strater, G. Alber and R. Hoffmann, ACS Chem. Biol., 2012, 7, 1281-1291.
5. F. Guida, M. Benincasa, S. Zahariev, M. Scocchi, F. Berti, R. Gennaro and A. Tossi, J. Med. Chem., 2015, 58, 1195-1204.
6. W. Li, J. Tailhades, N. O'Brien-Simpson, F. Separovic, L. Otvos, Jr., M. A. Hossain and J. Wade, Amino Acids, 2014, 46, 2287-2294.
7. G. Kragol, S. Lovas, G. Varadi, B. A. Condie, R. Hoffmann and L. Otvos, Jr., Biochemistry, 2001, 40, 3016-3026.
8. M. Scocchi, C. Liithy, P. Decarli, G. Mignogna, P. Christen and R. Gennaro, Int. J. Pept. Res. Ther., 2009, 15, 147-155.
9. A. Krizsan, D. Volke, S. Weinert, N. Strater, D. Knappe and R. Hoffmann, Angew Chem Int Ed, 2014, 53, 12236-12239.
10. A. C. Seefeldt, F. Nguyen, S. Antunes, N. Perebaskine, M. Graf, S. Arenz, K. K. Inampudi, C. Douat, G. Guichard, D. N. Wilson and C. A. Innis, Nat. Struct. Mol. Biol., 2015, 22, 470-475.
11. R. N. Roy, I. B. Lomakin, M. G. Gagnon and T. A. Steitz, Nat. Struct. Mol. Biol., 2015, 22, 466-469.
12. A. C. Seefeldt, M. Graf, N. Pérébaskine, F. Nguyen, S. Arenz, M. Mardirossian, M. Scocchi, D. N. Wilson and C. A. Innis, Nucleic Acids Res., 2016, DOI: 10.1093/nar/gkv1545.
13. E. Ostorhazi, M. C. Holub, F. Rozgonyi, F. Harmos, M. Cassone, J. D. Wade and L. Otvos, Jr., Int. J. Antimicrob. Agents, 2011, 37, 480-484.
14. W. Li, N. O'Brien-Simpson, J. Tailhades, N. Pantarat, R. Dawson, L. Otvos, Jr., E. Reynolds, F. Separovic, M. Hossain and J. Wade, Chem. Biol., 2015, 22, 1250-1258.
15. W. Li, M.-A. Sani, E. Jamasbi, L. Otvos Jr, M. A. Hossain, J. D. Wade and F. Separovic, Biochim. Biophys. Acta, Biomembr., 2016, 1858, 1236-1243.
16. W. Li, J. Tailhades, M. A. Hossain, N. M. O'Brien-Simpson, E. C. Reynolds, L. Otvos, F. Separovic and J. D. Wade, Aust. J. Chem., 2015, 68, 1373-1378.
17. L.-T. T. Nguyen, M. T. Gokmen and F. E. Du Prez, Polym. Chem., 2013, 4, 5527-5536.
18. J.-K. Y. Tan and J. G. Schellinger, Therapeutic Delivery, 2015, DOI: 10.4155/tde.15.52.
19. L. M. Tedaldi, M. E. B. Smith, R. I. Nathani and J. R. Baker, Chem. Commun., 2009, DOI: 10.1039/B915136B, 6583-6585.
20. M. E. B. Smith, F. F. Schumacher, C. P. Ryan, L. M. Tedaldi, D. Papaioannou, G. Waksman, S. Caddick and J. R. Baker, J. Am. Chem. Soc., 2010, 132, 1960-1965.
21. M. P. Robin, P. Wilson, A. B. Mabire, J. K. Kiviaho, J. E. Raymond, D. M. Haddleton and R. K. O'Reilly, J. Am. Chem. Soc., 2013, 135, 2875-2878.
22. J. Collins, J. Tanaka, P. Wilson, K. Kempe, T. P. Davis, M. P. McIntosh, M. R. Whittaker and D. M. Haddleton, Bioconjugate Chem., 2015, 26, 633-638.
23. E. Jamasbi, G. D. Ciccotosto, J. Tailhades, R. M. Robins-Browne, C. L. Ugalde, R. A. Sharples, N. Patil, J. D. Wade, M. A. Hossain and F. Separovic, Biochim. Biophys. Acta, Biomembr., 2015, 1848, 2031-2039.
24. G. B. Fields and R. L. Noble, Int. J. Pept. Protein Res., 1990, 35, 161-214.
25. S. Yao, D. K. Weber, F. Separovic and D. W. Keizer, Eur. Biophys. J., 2014, 43, 331-339.
26. P. T. Callaghan, M. A. Le Gros and D. N. Pinder, J. Chem. Phys., 1983, 79, 6372-6381.
27. S. Yao, G. J. Howlett and R. S. Norton, J. Biomol. NMR, 2000, 16, 109-119.
28. D. K. Wilkins, S. B. Grimshaw, V. Receveur, C. M. Dobson, J. A. Jones and L. J. Smith, Biochemistry, 1999, 38, 16424-16431.
29. M. Mohammed, A. Mohammed, M. Mirza and A. Ghori, Int. Res. J. Pharm, 2014, 5, 7-12.
30. G. M. Eldridge and G. A. Weiss, Bioconjugate Chem., 2011, 22, 2143-2153.
31. J. Youziel, A. R. Akhbar, Q. Aziz, M. E. B. Smith, S. Caddick, A. Tinker and J. R. Baker, Org. Biomol. Chem., 2014, 12, 557-560.
32. E. Glattard, E. S. Salnikov, C. Aisenbrey and B. Bechinger, Biophys. Chem., 2016, 210, 35-44.

1.-2. (canceled)

3. A kit comprising A3-APO or an analog, derivative, or oligomer thereof, or a pharmaceutically acceptable salt thereof; an antibiotic; and instructions for co-administering the A3-APO or an analog, derivative, or oligomer thereof, or a pharmaceutically acceptable salt thereof, and the antibiotic as part of a method for treating an infection.

4. The kit of claim 3 wherein the A3-APO or analog, derivative, or oligomer thereof, or a pharmaceutically acceptable salt thereof, and the antibiotic are each present in an amount individually subtherapeutic.

5. The kit of claim 3 wherein the dose of the A3-APO or an analog, derivative, or oligomer thereof, or a pharmaceutically acceptable salt thereof is within the hermetic zone or range.

6. The kit of claim 3 wherein the A3-APO or an analog, derivative, or oligomer thereof, or a pharmaceutically acceptable salt thereof, and the antibiotic are present in an amount in combination adapted for inhibiting toxin production by bacteria.

7. A method for treating a bacterial infection in a host animal, the method comprising administering to the host animal a therapeutically effective dose of a combination of A3-APO or analog, derivative, or oligomer thereof, or a pharmaceutically acceptable salt thereof; and an antibiotic.

8.-10. (canceled)

11. The method of claim 7 wherein the A3-APO or analog, derivative, or oligomer thereof, or a pharmaceutically acceptable salt thereof, and the antibiotic are each present in an amount individually subtherapeutic.

12. The method of claim 7 wherein the dose of the A3-APO or an analog, derivative, or oligomer thereof, or a pharmaceutically acceptable salt thereof is within the hermetic zone or range.

13. The method of claim 7 wherein the A3-APO or an analog, derivative, or oligomer thereof, or a pharmaceutically acceptable salt thereof, and the antibiotic are present in an amount in combination adapted for inhibiting toxin production by bacteria.

14.-15. (canceled)

16. The method of claim 7 wherein the host animal has or is at risk of having resistant bacteria.

17. The method of claim 7 wherein the A3-APO or analog, derivative, or oligomer thereof, or a pharmaceutically acceptable salt thereof is administered to the host animal prior to the antibiotic.

18. (canceled)

19. The method of claim 7 wherein the infection is caused at least in part by Gram-negative bacteria.

20. The method of claim 7 wherein the infection is caused at least in part by carbapenem-resistant Enterobacteriaceae (CRE).

21. The method of claim 7 wherein the infection is caused at least in part by extended spectrum beta-lactamase (ESBL) producing bacteria.

22. The method of claim 7 wherein the infection is a urinary tract infection.

23. The method of claim 7 wherein the infection is melioidosis.

24. The method of claim 7 wherein the infection is bacteremia.

25. The method of claim 7 wherein the infection is a wound infection.

26. The method of claim 7 wherein the infection is an infection associated with a prosthetic or device.

27. The method of claim 7 wherein the antibiotic is a polymyxin.

28. The method of claim 7 wherein the antibiotic is a beta-lactam antibiotic.
